(19) United States
(12) Patent Application Publication
(10) Pub. No.: US 2023/0390401 A1
(43) Pub. Date: Dec. 7, 2023

(54) RAPIDLY RELEASED BIOORTHOGONAL CAGING GROUPS

(71) Applicant: University of Utah Research Foundation, Salt Lake City, UT (US)

(72) Inventors: Raphael Franzini, Salt Lake City, UT (US); Julian Tu, Salt Lake City, UT (US)

(73) Assignee: University of Utah Research Foundation, Salt Lake city, UT (US)

(21) Appl. No.: 18/220,228

(22) Filed: Jul. 10, 2023

Related U.S. Application Data
(63) Continuation of application No. 17/059,163, filed on Nov. 25, 2020, now abandoned.
(60) Provisional application No. 62/704,007, filed on May 30, 2018.

(51) Int. Cl. A61K 47/54; A61K 31/27; A61K 31/352; A61K 31/407; A61K 31/4745; A61K 31/538; A61K 31/704; A61K 47/22

(52) U.S. Cl. CPC A61K 47/54; A61K 31/27; A61K 31/352; A61K 31/407; A61K 31/4745; A61K 31/538; A61K 31/704; A61K 47/22

(57) ABSTRACT

Bioorthogonal molecules are disclosed and described. A bioorthogonal a molecule having a structure according to:

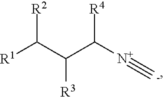

where R², R³, and R⁴ are independently selected from H, a substituted or unsubstituted C₁-C₄ alkyl or alkylene group, a substituted or unsubstituted aryl, COOR⁹, COR⁹, CONR⁹R¹⁰, CN, CF₃, SO₂R⁹, or a tether molecule;
R¹ is —R⁵, —OCOR⁶, —COR⁷, or —R⁸;
R⁵ is —R⁸, —OH, or tosyl;
R⁶ is a nitrophenyl ether or —R⁸;
R⁷ is —R⁸;
R⁸ is a payload or a molecular linker to a payload; and
R⁹ and R¹⁰ are independently selected from H or a substituted or unsubstituted C₁-C₄ alkyl or alkene group.

### CROSS-REFERENCE TO RELATED APPLICATIONS

This application is a continuation of U.S. patent application Ser. No. 17/059,163, filed Nov. 25, 2020, which is a 371 nationalization of PCT International Application No. PCT/US2019/034745, filed on May 30, 2019, which claims the benefit of U.S. Provisional Patent Application No. 62/704,007, filed on May 30, 2018, each of which is incorporated herein by reference in its entirety.

### BACKGROUND

Bioorthogonal chemistry generally refers to chemical reactions that can occur in biologic systems without interfering with native biochemical processes. Bioorthogonal chemistry provides reactions that are compatible with biomolecules, which facilitates the performance of chemistry in living organisms. Biocompatible reaction development has focused primarily on transformations that link two molecules, as such bioorthogonal ligation reactions have broad applicability in bioconjugation chemistry, materials science, and chemical biology. Such reactions have further been used to localize drugs and imaging agents at sites of disease.

### BRIEF DESCRIPTION OF THE DRAWINGS

FIG. 1 shows an illustration of a process for caging and uncaging an active molecule in accordance with an example of the present disclosure.

FIG. 2A illustrates an uncaging reaction of a caged molecule to release an active molecule in accordance with an example of the present disclosure.

FIG. 2B illustrates an uncaging reaction of a caged molecule to release an active molecule in accordance with an example of the present disclosure.

FIG. 3 shows chemical reactions used to generate three exemplary caging molecules in accordance with an example of the present disclosure.

FIG. 4A illustrates structures of caged 1,8-naphthalimide reporter probes and products of their reactions with tetrazine in accordance with an example of the present disclosure.

FIG. 4B illustrates data related to tetrazine-mediated ICPr/ICPrc-group removal in accordance with an example of the present disclosure.

FIG. 5A shows molecular structures of caged cancer therapy agents in accordance with an example of the present disclosure.

FIG. 5B illustrates data relating to release percentages of cancer therapy agents from FIG. 5A following uncaging in accordance with an example of the present disclosure.

FIG. 5C illustrates data relating to EC₅₀ values in cytotoxicity studies for the cancer therapy agents from FIG. 5A in accordance with an example of the present disclosure.

FIG. 6 shows the measurement of cytotoxicity of reaction-activated doxorubicin (upper panel) and mitomycin C (lower panel) in A549 lung adenocarcinoma cells in accordance with an example of the present disclosure.

FIG. 7A shows the structures of ICPr-modified resorufin (ICPr-rsf) and ICPrc-modified mexiletine (ICPrc-mex) along with an illustration of experiments involving implantation into zebrafish embryos in accordance with an example of the present disclosure.

FIG. 7B shows image detection of resorufin fluorescence upon tetrazine-mediated uncaging in zebrafish in accordance with an example of the present disclosure.

FIG. 7C shows data relating to fluorescence increase in zebrafish with either Tz-PS or unmodified beads in when incubated with ICPr-rsf in accordance with an example of the present disclosure.

FIG. 7D shows data relating to the decrease in heart rate in zebrafish implanted with either Tz-PS or unmodified beads treated with ICPrc-mex in accordance with an example of the present disclosure.

### DETAILED DESCRIPTION

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only, and is not intended to be limiting. Unless defined otherwise, all technical and scientific terms used herein have the same meaning as commonly understood by one of ordinary skill in the art to which this disclosure belongs. Also, the same reference numerals in appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein, but are merely representative thereof. One skilled in the relevant art will also recognize that the technology can be practiced without one or more of the specific details, or with other methods, components, layouts, etc. In other instances, well-known structures, materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 5% in some examples, less than 1% in other examples, and less than 0.01% in yet other examples.

The term “dosage unit” or “dose” are understood to mean an amount of an active agent that is suitable for administration to a subject in order achieve or otherwise contribute to a therapeutic effect. In some examples, a dosage unit can refer to a single dose that is capable of being administered to a subject or patient, and that may be readily handled and packed, remaining as a physically and chemically stable unit dose.

As used herein, a “dosing regimen” or “regimen” such as “treatment dosing regimen,” or a “prophylactic dosing regimen” refers to how, when, how much, and for how long a dose of an active agent or composition can or should be administered to a subject in order to achieve an intended treatment or effect.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “therapeutic agent,” “active agent,” and the like can be used interchangeably and refer to an agent that can have a beneficial or positive effect on a subject when administered to the subject in an appropriate or effective amount.

The phrase “effective amount,” “therapeutically effective amount,” or “therapeutically effective rate(s)” of an active ingredient refers to a substantially non-toxic, but sufficient amount or delivery rates of the active ingredient, to achieve therapeutic results in treating a disease or condition for which the drug is being delivered. It is understood that various biological factors may affect the ability of a substance to perform its intended task. Therefore, an “effective amount,” “therapeutically effective amount,” or “therapeutically effective rate(s)” may be dependent in some instances on such biological factors. Further, while the achievement of therapeutic effects may be measured by a physician or other qualified medical person using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

As used herein, “formulation” and “composition” can be used interchangeably and refer to a combination of at least two ingredients. In some embodiments, at least one ingredient may be an active agent or otherwise have properties that exert physiologic activity when administered to a subject. For example, amniotic fluid includes at least two ingredients (e.g. water and electrolytes) and is itself a composition or formulation.

As used herein, a “subject” refers to an animal. In one aspect the animal may be a mammal. In another aspect, the mammal may be a human.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

Concentrations, amounts, and other numerical data may be presented herein in a range format. It is to be understood that such range format is used merely for convenience and brevity, and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

An initial overview of embodiments is provided below, and specific embodiments are then described in further detail. This initial summary is intended to aid readers in understanding the disclosure more quickly but is not intended to identify key or essential technological features, nor is it intended to limit the scope of the claimed subject matter.

Bioorthogonal chemistry provides biomolecule-compatible reactions capable of being performed in living organisms. Biocompatible reaction development has focused primarily on transformations that link two molecules, as such bioorthogonal ligation reactions have broad applicability in bioconjugation chemistry, materials science, and chemical biology. Such reactions can be used to localize drugs and imaging agents at specific locations, such as sites of disease. In contrast, the discovery of bioorthogonal cleavage reactions that allow for the controlled release of payloads has only recently attracted substantial research interest, even though such reactions are valuable in a wide range of applications. One potential reason may be due to the scarcity of bioorthogonal bond-cleavage reactions, which remains a bottleneck to the advancement of reaction-based applications in chemical biology and smart therapeutics. Until recently, modified Staudinger reactions were the only bioorthogonal release reactions available.

The development of the inverse-electron demand Diels-Alder (IEDDA) pyridazine elimination reaction between carbamate-modified trans-cyclooctenes (TCO) and Tz was a breakthrough in this regard. The rate of this reaction was significantly faster than the Staudinger reaction and it obviated the use of metabolically unstable phosphines, allowing for widespread use in chemical biology. Other examples of bioorthogonal cleavage reactions include the strain-promoted 1,3-dipolar cycloaddition of TCO and p-azidobenzylcarbamates and the Tz-mediated removal of vinyl ethers. However, reactions generally need to meet several strict requirements for in vivo use, including rapid reaction rate, near-quantitative payload release, non-toxic reagents, and extended serum stability. Currently available reactions only partially meet these conditions.

Accordingly, the present disclosure provides various bioorthogonal molecules and uses for such molecules that address many, if not all, of these concerns. As a general example, certain 3-isocyanopropyl substituents can function as masking groups that can be effectively removed in biologic environments and systems.

The ability to effectively mask and chemically control the release of reporter probes, bioactive compounds, and biomacromolecules using bioorthogonal reactions is opening up opportunities for the development of innovative research tools, diagnostics, and therapeutics. Such dissociative bioorthogonal reactions allow for the effective masking and chemically controlled release of numerous payload moieties such as, without limitation, bioactive agents, prodrugs, antibodies, cytotoxic agents, reporter probes, pharmacophores, biomacromolecules, and the like, thus allowing opportunities for innovative research tools, diagnostics, and therapeutics, to name a few. Application in diverse fields including biosensing, cell imaging, gaso-emission, activity control of nucleic acids and proteins, and the like exemplify the potential of such transformations. The clinical use of dissociative bioorthogonal reactions in chemically responsive spatiotemporal reporter probe and/or drug targeting can be of great use in the diagnosis and/or treatment of spatially localized medical conditions. For example, antibody-reporter conjugates may allow the location of such a medical condition to be identified with a high degree of specificity. Subsequent treatment of the medical condition with antibody-drug conjugates can be similarly targeted to specific identified locations. Such targeted drug deliver can treat the medical condition more effectively due to the localized aggregation of the drug at the specific identified location prior to drug activation, which can also reduce many drug-induced side effects by minimizing contact of the active drug with unaffected tissues, organs, and the like.

Dissociative bioorthogonal reactions can be used in proximity-reporting analytical probes for sensing biomarkers, proteins, externally introduced compounds, and the like, in cells, plants, animals, and the like. Further example applications can include DNA sequencing, enzyme uncaging, cell imaging, biomacromolecule purification, multiplexed in situ protein detection, and as ultra-mild protecting groups, to name a few. Applications of dissociative in vivo chemistry leading to spatiotemporally controlled release of drugs are particularly appealing because of the potential for clinical translation in, for example, cancer treatments such as chemotherapy. For example, implantation of tetrazine (Tz)-modified biomaterials can facilitate the localized activation of prodrugs (e.g. doxorubicin), potentially providing significantly more potent anti-tumor effects relative to systemic doxorubicin administration, which can also reduce side-effects. In another example approach, bioorthogonal reactions can be designed to activate antibody-drug conjugates in vivo. In this pre-targeting strategy, an antibody conjugated with a drug via a chemically cleavable linker can accumulate at a desired site, and the subsequent administration of a trigger or cleavage molecule can liberate the drug specifically in the target tissue.

The present disclosure shows that the reaction is rapid and can liberate, for example, phenols and amines near-quantitatively under physiological conditions. The reaction is compatible with living organisms as demonstrated by the release of a resorufin fluorophore and a mexiletine drug in zebrafish embryos implanted with tetrazine-modified beads. The combined benefits of synthetic ease, rapid kinetics, diversity of leaving groups, high release yields, and structural compactness, make 3-isocyanopropyl derivatives attractive chemical caging moieties for uses in chemical biology, drug delivery, and the like.

Nonlimiting examples of bioorthogonal chemistries can include Staudinger reactions, inverse-electron demand Diels-Alder cycloadditions, borane-induced deoxygenations, strain-promoted [3+2] azide-alkyne cycloadditions, metal-catalyzed uncaging, and the like. The utility of several of these reactions to control the release of drugs and fluorophores has been demonstrated in vitro and in living vertebrates. To further advance the scope of bond-cleavage reactions compatible with biologic systems, the present disclosure also provides structurally compact bioorthogonal reagents that are facile to synthesize while meeting the key requirements of rapid reaction kinetics, high release yields, broad range of leaving groups, lack of toxicity, and extended serum stability.

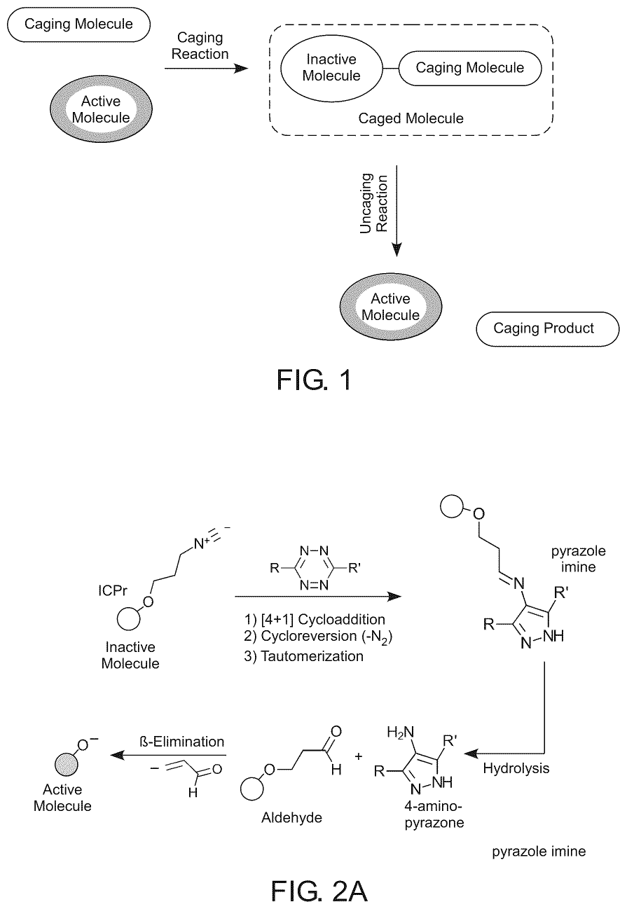

As used in the present disclosure, the term “bioorthogonal molecule” can refer to a caging molecule capable of blocking functionality of a molecule/payload or a caged molecule having a molecule/payload coupled to a caging molecule. FIG. 1, for example, initially shows an active molecule and a caging molecule on the left. A caging reaction can be used to couple the caging molecule to a functional group of the active molecule, thus blocking functionality and causing the active molecule to become an inactive molecule. The inactive molecule with the caging molecule coupled to the functional group is referred to herein as a “caged molecule.” The caging molecule can be released from the functional group of the inactive molecule by an uncaging reaction, which thus restores functionality to the now active molecule. It is noted that, depending on the specifics of the caged molecule and/or the caging reaction, one or more intermediates can be generated before the caging molecule is released from the active molecule. Any intermediate coupled to the caging molecule that remains in an inactive state with respect to the caged functional group is additionally considered to be a caged molecule. FIG. 1 additionally shows a caging product released from the caged molecule from the uncaging reaction. In some examples, the uncaging reaction can be a bioorthogonal cleavage or other bioorthogonal reaction that is compatible with biologic environments, cells, and living organisms. While such caging/uncaging reactions are described herein in terms of biologic compatibility, such is not intended to be limiting, and it is understood that the present disclosure includes uses of these materials/reactions in non-biologic environments.

The present disclosure describes various bioorthogonal molecules that can generally have a structure according to Structure 01:

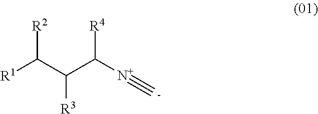

where R², R³, and R⁴ can be independently selected from H, a substituted or unsubstituted C₁-C₄ alkyl or alkylene group, a substituted or unsubstituted aryl, COOR⁹, COR⁹, CONR⁹R¹⁰, CN, CF₃, SO₂R⁹, a tether molecule, or the like. In one example, R⁹ and R¹⁰ can be independently selected from H or a substituted or unsubstituted C₁-C₄ alkyl or alkylene group. R¹ can be one of —R⁵, —OCOR⁶, —COR⁷, or —R⁸, where R⁸ can be —R⁸, —OH, or tosyl, R⁶ can be a nitrophenyl ether or —R⁸, R⁷ is —R⁸, and R⁸ is a payload or a molecular linker (linker) to payload.

One general example where R¹ is R⁵, R⁵ is R⁸, and R², R³, and R⁴ are H is shown according to Structure 02:

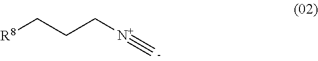

Structure 02 is generally referred to herein as 3-isocyanopropyl (ICPr), which can include both caging molecules and caged molecules.

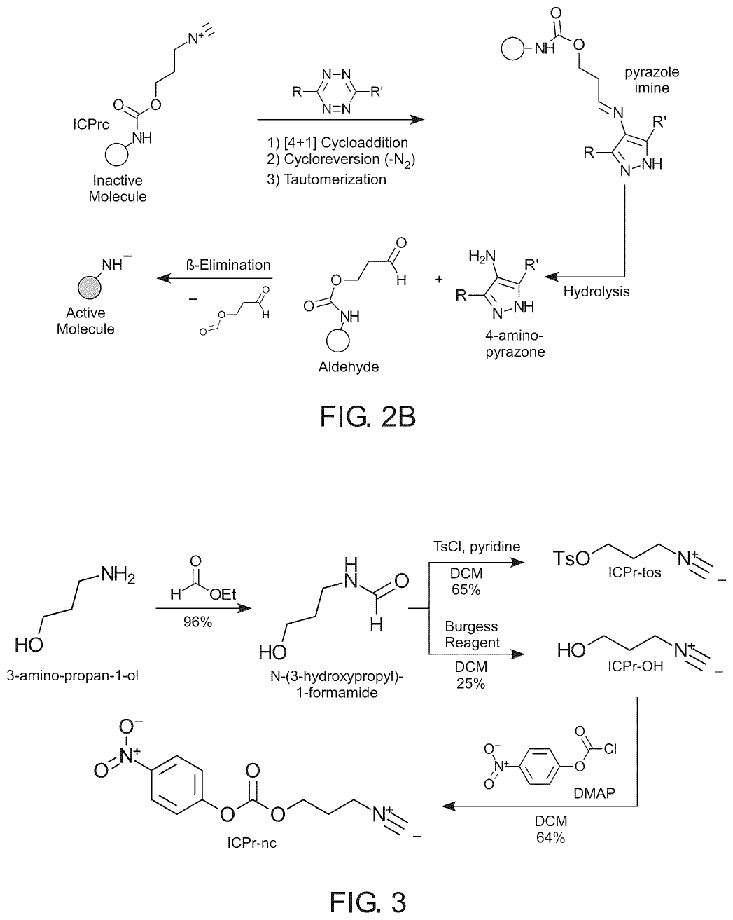

FIG. 2A shows one nonlimiting example of a caging release reaction where, initially, an ICPr is shown coupled to a molecule (or payload) at an —O functional group that has inactivated the payload molecule. The ICPr is contacted with a tetrazine, such as a 1,2,4,5-tetrazine, to initiate the uncaging reaction. When brought into contact, tetrazines convert isonitriles to aldehydes at temperatures compatible with standard biologic and physiologic environments. More specifically, and without intending to be bound to any chemical theory, the ICPr molecule undergoes a [4+1] cycloaddition reaction when in contact with a tetrazine. This is followed by a rapid expulsion of N2 due to cycloreversion and the subsequent formation by tautomerization of a pyrazole imine intermediate. The pyrazone imine intermediate hydrolyzes to an aldehyde coupled to the inactive payload molecule and an amino pyrazone (e.g., 4-amino-pyrazone). Due to the acidity of the aldehyde's α-proton, the payload molecule is spontaneously released via β-elimination as a leaving group from the C-1 position of the 3-oxypropyl moiety. It is noted that the R and R′ groups on the tetrazine molecule shown in FIG. 2A are meant to represent any tetrazine capable of initiating an uncaging reaction.

Another general example where R¹ is —OCOR⁶, R⁶ is R⁷, and R², R³, and R⁴ are H is shown according to Structure 03:

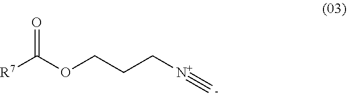

Structure 3 is generally be referred to herein as 3-isocyanopropyl-1-carbamonyl (ICPrc) where R⁷ is —N. ICPrc can include both caging molecules and caged molecules, specific examples of which follow below.

FIG. 2B shows one nonlimiting example of a caging release reaction where, initially, an ICPrc is shown coupled to a molecule (or payload) at an —NH functional group that has inactivated the payload molecule. The ICPrc is contacted with a tetrazine, such as a 1,2,4,5-tetrazine, to initiate the uncaging reaction. When brought into contact, tetrazines convert isonitriles to aldehydes at temperatures compatible with standard biologic and physiologic environments. More specifically, and without intending to be bound to any chemical theory, the ICPrc molecule undergoes a [4+1] cycloaddition reaction when in contact with a tetrazine. This is followed by a rapid expulsion of N2 due to cycloreversion and the subsequent formation by tautomerization of a pyrazole imine intermediate. The pyrazone imine intermediate hydrolyzes to an aldehyde coupled to the inactive payload molecule and an amino pyrazone (e.g., 4-amino-pyrazone). Due to the acidity of the aldehyde's α-proton, the payload molecule is spontaneously released via β-elimination as a leaving group from the C-1 position of the 3-oxypropyl moiety. It is noted that the R and R′ groups on the tetrazine molecule shown in FIG. 2B are meant to represent any tetrazine capable of initiating an uncaging reaction.

Numerous caging molecules can be utilized to cage, and thus inactivate, a payload molecule as a caged molecule that can be released via bioorthogonal cleavage. Various isonitrile molecules, for example, can be used to inactivate diverse payload molecules by caging to generate a caged molecule, which can subsequently be effectively removed by bioorthogonal reactions with cleavage agents such as tetrazines (e.g., 1,2,4,5-tetrazines). One example of an isonitrile caging molecule is 3-isocyanopropan-1-ol (ICPr-OH), where R², R³, and R⁴ are H and R¹ is —OH according to Structure 04:

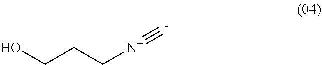

In another example of an isonitrile caging molecule is 3-isocyano-1-tosylpropane (ICPr-tos), where R², R³, and R⁴ are H and R¹ is tosyl according to Structure 05:

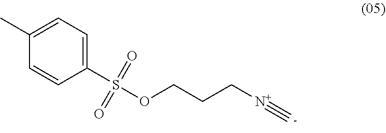

ICPr-tos can be used for, among other things, the alkylation of phenols and other nucleophiles.

In another example of an isonitrile caging molecule is 3-isocyanopropyl-1-(4-nitrophenyl)carbonate (ICPr-nc), where R², R³, and R⁴ are H, R¹ is —OCOR⁶, and R⁶ is 4-nitrophenyl according to Structure 06:

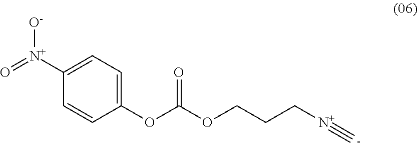 [SMILES: [C-]#[N+]CCCOC(=O)Oc1ccc([N+](=O)[O-])cc1]

ICPr-nc can be used for, among other things, masking amines.

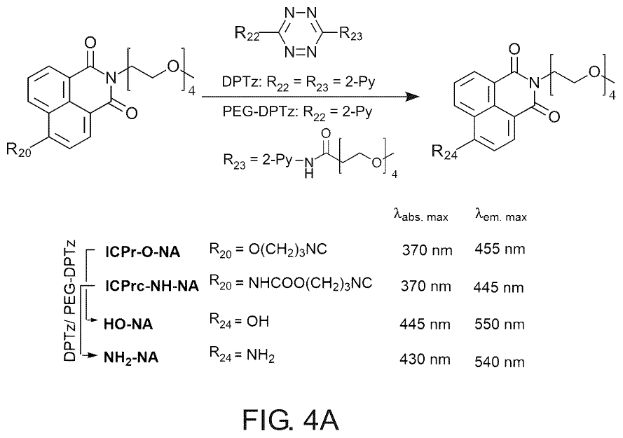

FIG. 3 shows exemplary syntheses for ICPr-OH, ICPr-tos, and ICPr-nc, which are described in more detail in the examples section below. Generally, however, 3-amino-propan-1-ol is reacted with ethyl formate to form N-(3-hydroxypropyl)-1-formamide. In the upper reaction, tosyl chloride in dichloromethane (DCM) is reacted with the N-(3-hydroxypropyl)-1-formamide intermediate in pyridine under nitrogen atmosphere to generate ICPr-tos. In the lower reaction, Burgess Reagent (methyl N-(triethylammoniumsulfonyl)carbamate) is reacted with the N-(3-hydroxypropyl)-1-formamide intermediate in DCM and under nitrogen atmosphere to form ICPr-OH. To generate the third caging molecule shown in FIG. 3, 4-(dimethylamino)pyridine (DMAP) and nitrophenyl chloroformate are reacted with ICPr-OH in DCM to form ICPR-nc. It is noted that ICPr-nc tends to decompose gradually over time. As such, it can be beneficial to utilize the molecule soon after synthesis.

In other examples, bioorthogonal molecules can have a variety of R², R³, and R⁴ groups depending on the nature of the payload, the delivery mechanism, a target location, and the like. In one example, at least one of R², R³, or R⁴ can be a substituted or unsubstituted aryl, such as a phenyl. In one example of such a caging molecule is 3-isocyano-2-phenylpropan-1-ol, where R² and R⁴ are H, R³ is phenyl, and R¹ is —OH according to Structure 07:

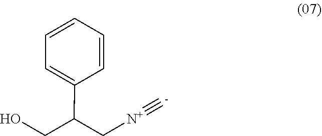

Various isonitrile caging molecules that include tosyl groups are also considered, in addition to the Structure 05 moiety. Nonlimiting examples can include isonitrile molecules where at least one of R², R³, or R⁴ is not H and R¹ is tosyl. One specific example is 3-isocyano-2-phenyl-1-tosylpropane, where R² and R⁴ are H, R³ is phenyl, and R¹ is tosyl according to Structure 08:

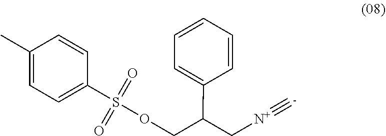

Yet another example of an isonitrile caging molecule with some similarities to Structure 07 is 3-isocyano-1-phenylpropan-1-ol, where R² is phenyl, R³ and R⁴ are H, and R¹ is —OH according to Structure 09:

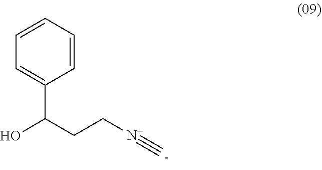

As described above, in some examples R⁸ can be a linker to a payload, which can include a leaving group. Nonlimiting examples of such leaving groups can include esters, carbonates, aromatic esters, phosphates and phosphate derivatives, hydroxamate esters, ammonium compounds, and the like. Thus, in some examples, the leaving group can include COOR¹¹, O-Aryl-R¹¹, POR¹¹R¹²R¹³⁺, ONHOR¹¹, or NR¹¹R¹²R¹³⁺, wherein R¹¹, R¹² and R¹³ are independently selected from a second leaving group (e.g. a payload, a substrate, a reporter molecule, etc.), H, and a substituted or unsubstituted C₁-C₄ alkyl or alkylene group. In some specific examples, the leaving group can include COOR¹¹ or POR¹¹R¹²R¹³⁺, wherein R¹¹, R¹², and R¹³ are independently selected from a second leaving group (e.g. a payload, a substrate, a reporter molecule, etc.), H, and a substituted or unsubstituted C₁-C₄ alkyl or alkylene group.

Caging molecules can be used to cage and thus block the functionalities of diverse payload molecules. The functional activity of a given payload molecule can be restored by an uncaging reaction that disrupts the caging molecule and releases the payload molecule. Any molecular material capable of being caged and subsequently released by an uncaging reaction is considered to be within the present scope. In some examples, the payload molecule can be compatible with bioorthogonal reaction chemistry. Nonlimiting examples of general groups of payload molecules can include bioactive agents, therapeutic agents such as drugs, prodrugs, metabolites, and the like, cytotoxic agents and cytotoxic materials, nutritional supplements, vitamins, reporter molecules, affinity binders such as antibodies, biotin and biotin derivatives, and the like, pharmacophores, biomolecules, biomacromolecules, polymers, and the like, including combinations thereof.

In one example, a payload molecule can be a reporter molecule. Any useful reporter molecule that is capable of being inactivated by a caging group is considered to be within the present scope. Nonlimiting examples can include chromophores, fluorophores, profluorophores, luminophores, chemiluminophores, dyes, radionuclides, and the like, including combinations thereof. One example of a reporter molecule can include a 1,8-naphthalimide derivative according to Structure 10:

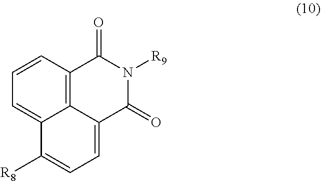

where R₈ can be —OH or NH₁₂ and R₉ can be a polymer, a molecular tether, or the like, provided R₉ does not negatively interfere with the florescent reporting of the molecule or the caging process. Polymers can be utilized for various reasons, such as adjusting the solubility of the naphthalimide molecule. In one example, R₉ can be a polyethylene glycol (PEG) polymer to make the naphthalimide molecule more soluble in aqueous environments. A general example of such a molecule is shown according to Structure 11:

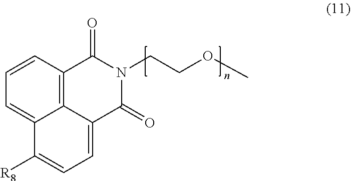

Examples of caged 1,8-naphthalimide molecules are represented as Structures 12-16 in the example synthesis section below. Another specific example of a reporter molecules can include resorufin, caged as 3-isocyanopropyl resorufin ether (ICPr-rsf, Structure 17), also in the example synthesis section below.

In another example, a therapeutic agent, bioactive agent, vitamin, nutritional supplement, cytotoxic agents, cytotoxic material, or the like, can be caged and inactivated according to the present disclosure. Any such molecule or material that can be inactivated by caging and reactivated upon release is considered to be within the present scope. Nonlimiting examples include heart-related medications such as mexiletine, caged as N-(3-isocyanopropyl-1-carbamoyl)mexiletine (ICPrc-mex, Structure 21) and coumarin, caged as 7-(3-isocyanopropyl-1-oxy)-coumarin (ICPr-coum, Structure 18), and cancer therapeutics such as Mitomycin C, caged as N-(3-isocyanopropyl-1-carbamoyl)Mitomycin C (ICPrc-mmc; Structure 19), doxorubicin, caged as N-(3-isocyanopropyl-1-carbamoyl)doxorubicin (ICPrc-dox; Structure 20), SN-38, caged as N-(3-isocyanopropyl-1-carbamoyl)SN-38 (ICPr-SN-38, Structure 22), mercaptopurine, caged as 6-(3-isocyanopropyl)mercaptopurine (ICPr-mp; Structure 23), 2-naphthalenethiol, caged as 2-(3-isocyanopropyl-2-phenyl)Naphthalenethiol (Structure 24), and the like. Each of these structures can be found in the example synthesis section below. It should be noted for these and other caged molecules disclosed herein, the specific structures shown are merely exemplary and are not limiting. Other structural caging configurations, caging molecules, and/or caging groups can be substituted that retain the caging/uncaging functionality, and all such alterations are within the present scope.

Returning to the general example of Structure 1, in some cases at least one of R², R³, and R⁴ can be a tether, which can be chemically modified or conjugated as desired. The tether can be attached directly to the carbon backbone or through another molecule, such as one of the substitution groups described for R², R³, and R⁴. For example, one of R², R³, and R⁴ can be an aryl such as phenyl, to which the tether can be attached.

A tether can link the bioorthogonal molecule to a variety of substrates, such as a biomolecule (e.g. glutathione, serum albumin, immunoglobulin, DNA, RNA, antibody, or the like), a homing molecule (e.g. small-molecule ligand, peptide, polypeptide, aptamer, or the like), a macromolecule (e.g. polymer, dendrimer, micelle, or the like), a releasing molecule, a caging molecule, a caged molecule, and the like, including combinations thereof.

In one nonlimiting example, the tether can be —SR¹⁴, were R¹⁴ can be a substituted or unsubstituted C₁-C₄ alkyl or alkylene group, a biomolecule (e.g. glutathione, serum albumin, immunoglobulin, DNA, RNA), or the like. In some examples, R¹⁴ can be a homing molecule (e.g. small-molecule ligand, peptide, polypeptide, aptamer) and the homing molecule can be linked to the bioorthogonal molecule either directly or via a tether. In other examples, R¹⁴ can be a material or macromolecule (e.g. polymer, dendrimer, micelle).

The present bioorthogonal molecules can also be incorporated into oligonucleotides (e.g. DNA, RNA) or derivatives thereof (e.g. PNA, LNA, 2′-OMe-RNA, phosphorothioates) as, for example, one or more bioorthogonal molecules as modified nucleobases, at the termini, and/or within the backbone. For example, caging groups can be directly attached to the specified residues or via immolative spacers. As such, payload molecules can be attached to the oligonucleotide as caged molecules and released by a subsequent uncaging reaction. The following structures represent some examples of attachment points for a bioorthogonal molecule (BNBD) to a nucleic acid. For example, in some cases, the bioorthogonal molecule can be attached directly to the specific nucleobase as follows:

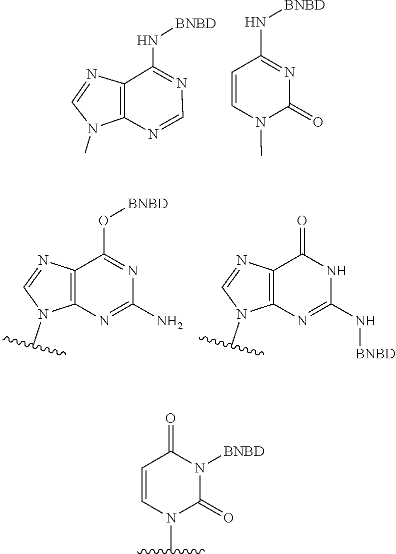

In other examples, the bioorthogonal molecule can be attached to a nucleic acid at the backbone, such as in the following structure:

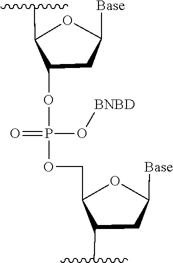

Regardless of the particular attachment point, bioorthogonal molecules can be used in a variety of methods employing nucleic acids. For example, in some cases, the bioorthogonal molecules can be used in methods to reconstitute the structure of an oligonucleotide or a polynucleotide by contacting the bioorthogonal molecule with a releasing molecule to release the underlying oligonucleotide. Specifically, a variety of bioorthogonal molecules can be attached to a nucleic acid (e.g. an oligonucleotide or a polynucleotide) to form a modified nucleic acid. As desired, the bioorthogonal molecule can be reacted with a releasing group to produce a reconstituted nucleic acid.

Such bioorthogonal molecules can also be used in methods to control the hybridization of oligonucleotides or polynucleotides via removal of bioorthogonal molecule modifications using a releasing molecule to release the target nucleic acid. Specifically, a target nucleic acid can be prevented from hybridizing with a modified nucleic acid probe due to a removable coupling of a bioorthogonal molecule to the nucleic acid probe. Reaction with a releasing group can remove the bioorthogonal molecule from nucleic acid probe to allow hybridization and to prepare a reconstituted nucleic acid.

Similarly, the bioorthogonal molecules can also be used in methods to control the folding of oligonucleotides or polypeptides by removal of bioorthogonal molecule modifications by reaction with a releasing molecule. For example, a modified nucleic acid can be prevented from folding due to the presence of bioorthogonal molecules removably coupled thereto. Reaction with a releasing group can remove the bioorthogonal molecules from the nucleic acid to allow proper folding of the nucleic acid and preparation of a reconstituted nucleic acid. In some examples, the nucleic acid (e.g. an oligonucleotide) can be an aptamer or ribozyme.

In some examples, the bioorothogonal molecules can be used in methods for the synthesis of BNBD-modified oligonucleotides by reacting phosphorothioates with bioorthogonal molecules having a suitable leaving group. In some cases, the bioothogonal molecules can be used in methods for the synthesis of modified oligonucleotides by incorporation of modified nucleotide derivatives during oligonucleotide solid phase synthesis. For example, the bioorthogonal molecules can be precursors for the solid-phase synthesis of modified oligonucleotides (for example by phosphite or phosphoramidite method). In some examples, the precursors can also be modified on the nucleobase. One non-limiting example is depicted below.

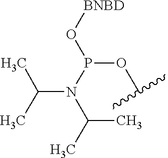

Additionally, the bioorthogonal molecules can also be used in methods that remove bioorthogonal molecule modifications from oligonucleotide backbones by reaction with a releasing molecule. In some examples, the backbone can have a phosphate or phosphorothioate structure. In some cases, bioorthogonal molecules can be used in methods for the removal of a bioorthogonal molecule from an oligonucleotide terminus by reaction with a suitable releasing molecule. Methods can include multiple cycles of incorporating a nucleotide containing a modification of the bioorthogonal molecule and removal of the bioorthogonal molecule by contact with a suitable releasing molecule.

In some examples, bioorthogonal molecules can be used to control the dissociation of oligonucleotides through the selective removal of one or more bioorthogonal molecule modifications from the backbone. Specifically, a modified nucleic acid can include bioorthogonal molecule modifications that modify the stability of the modified nucleic acid to increase or decrease the dissociation rate of nucleic acid strands. Reaction with a releasing group can remove the bioorthogonal molecule modification(s) from the modified nucleic acid to alter stability, thus increasing or decreasing the stability of associated nucleic acid strands.

In some additional examples, bioorthogonal molecules can be used for cell delivery of oligonucleotides and intracellular activation of oligonucleotides. Such techniques can include modifying an oligonucleotide with the bioorthogonal molecule to increase its permeability to a membrane that may be otherwise impermeable to the free oligonucleotide (e.g. cell membrane). Removal of the modifications by contact with a releasing molecule once inside a cell or organelle can thus decrease the membrane permeability of the oligonucleotide and increase retention in the cell or organelle.

In some examples, bioorthogonal molecules can be used in methods to elucidate the composition of an oligonucleotide molecule (e.g. DNA sequencing) by sequential incorporation of one or several nucleotides resulting in the modification of one of the termini (such as the 3′ terminus, for example) with a bioorthogonal molecule, a reading step, and removal of the modification.

In other examples, bioorthogonal molecules can be used in methods for the detection of an analyte or target molecule (such as a biomacromolecule, for example) where the release of a caging group is linked to a reporter signal (e.g. fluorescence turn-on, activation of MRI contrast agent, chemiluminescence signal, bioluminescence signal, etc.). In some examples, the bioorthogonal molecule and target molecule can include a quencher/fluorophore pair.

In some examples, a target molecule can be a biomarker. In some examples, the bioorthogonal molecules can be used in methods for delivering or localizing a therapeutic agent or reporter molecule in which a homing molecule that binds to a biomarker is modified with an affinity binder that includes a releasing molecule to cause uncaging at the biomarker/homing molecule. In some examples, the affinity binder can be an antibody specific to the caged molecule.

In some examples, bioorthogonal molecules can be used in methods for delivering or localizing a therapeutic agent or reporter molecule in which proximal binding of two homing molecules reveal a template molecule that can be targeted by compositions as described herein.

In some examples, bioorthogonal molecules can be used in methods of spatiotemporally controlled release of therapeutics or imaging agents in which a caged molecule of a therapeutic or an imaging agent is co-administered simultaneously or sequentially with a releasing molecule targeted in a location or time-dependent manner. In other examples, bioorthogonal molecules can be used in methods of spatiotemporally controlled release of therapeutics or imaging agents in which caged molecules are targeted to accumulate at specific locations such as, but not limited to, a specific tissue (e.g. mucosal tissue), a specific medical condition (e.g. tumor), or organ (e.g. bladder, kidney, liver).

In some examples, bioorthogonal molecules can be used in methods of delivering molecules into a cell or other structure (e.g. organelle) with an impermeable or partially permeable membrane, in which the molecule of interest is modified with moieties of the bioorthogonal molecule to be permeable to the membrane (e.g. plasma membrane). In a subsequent step, contact with releasing molecules can remove the bioorthogonal molecules, which can increase retention of the molecules of interest within the membrane interior (i.e. inside the cell) due to the reduced membrane permeability.

In some examples, target molecules can include a carrier molecule (e.g. protein, oligonucleotide, colloid, nanoparticle, liposome, micelle, dendrimer, surface, polymer, viral particle, cell surface, hydrogel, small molecule) modified with one or more bioorthogonal molecules conjugated either directly or via a tether. In some examples, the carrier molecule leads to accumulation at a specific anatomical localization (e.g. tissue, organ) and/or endows beneficial pharmacokinetic properties. Multiple bioorthogonal molecules with the same or different caged molecules can be attached to one or more carrier molecules. In one example, two or more different therapeutic agents can be attached to a single carrier molecule. In another example, the target molecule (via multiple bioorthogonal molecules) can include both releasable therapeutic agents and releasable reporter molecules.

The bioorthogonal molecule can also be included in a therapeutic composition. The therapeutic composition can include an effective amount, or a therapeutically effective amount, of a therapeutic agent coupled as a payload to the biorthogonal molecule in a pharmaceutically acceptable carrier. As will be appreciated by one skilled in the art, the effective amount, or therapeutically effective amount, can be highly dependent on the particular therapeutic agent linked to the bioorthogonal molecule. Further a variety of therapeutic agents can be linked to the biorthogonal molecule, examples of which have been described herein. Nonlimiting examples of possible therapeutic agents can include doxorubicin, auristatins, mitomycin C, coumarin, mexiletine, SN-38, mercaptopurine, 2-naphthalenethiol, and the like. The nature of the pharmaceutically acceptable carrier can depend on the intended mode of administration. For example, the pharmaceutically acceptable carrier can be formulated to administer the therapeutic composition via injection, enteral administration, topical administration, transdermal administration, transmucosal administration, inhalation, implantation, or the like.

In some examples, the pharmaceutically acceptable carrier can be formulated to provide a therapeutic composition for administration via injection, such as intramuscular injection, intravenous injection, subcutaneous injection, intradermal injection, intrathecal injection, intraocular injection, or the like. In such examples, the pharmaceutically acceptable carrier can include a variety of components, such as water, a solubilizing or dispersing agent, a tonicity agent, a pH adjuster or buffering agent, a preservative, a chelating agent, a bulking agent, the like, or a combination thereof.

In yet other examples, the pharmaceutically acceptable carrier can be formulated to provide a therapeutic composition for enteral administration, such as via solid oral dosage forms or liquid oral dosage forms. In the case of solid oral dosage forms, the pharmaceutically acceptable carrier can include a variety of components suitable for forming a capsule, tablet, or the like. In the case of a liquid dosage form, the pharmaceutically acceptable carrier can include a variety of components suitable for forming a dispersion, a suspension, a syrup, an elixir, or the like.

In yet other examples, the pharmaceutically acceptable carrier can be formulated to provide a therapeutic composition for topical, transdermal, or transmucosal administration, such as to the skin, to the eye, to the vaginal cavity, to the rectum, to the nasal cavity, the like, or a combination thereof. Further, the topical, transdermal, or transmucosal formulations can be formulated for local and/or systemic delivery of one or more components of the therapeutic composition.

In some additional examples, the pharmaceutically acceptable carrier can be formulated for administration via inhalation. In some examples, such formulations can include a propellant, such as hydrofluoralkanes, such as HFA134a, HFA227, or other suitable propellant. In yet other examples, the therapeutic composition can be formulated for administration via nebulization. In either case, the therapeutic composition can also include a variety of solubilizing agents, such as those described above. In other examples, the therapeutic composition can be formulated as a dry powder aerosol. In some examples, the therapeutic composition can include a particulate carrier and/or other particulate excipients, such as lactose, mannitol, other crystalline sugars, fumed silica, magnesium stearate, amino acids, the like, or combinations thereof.

In some specific examples, the pharmaceutically acceptable carrier can be formulated to provide a therapeutic composition for ocular administration. Non-limiting examples can include topical application to the eye in the form of a drop, a gel, a film, an insert, a sponge, an ointment, the like, or a combination thereof. In yet other examples, the therapeutic composition can be formulated for intraocular injection or implantation in the form of a solution, a depot, a scaffold, the like, or a combination thereof.

It is noted that a variety of components are listed for use in specific carriers or carrier types. However, the lists of components are not necessarily intended to be exclusive to a particular carrier or carrier type. For example, the biodegradable polymers specifically listed with reference to ocular formulations may also be useful in other carriers or carrier types as well. Thus, where suitable, any of the components disclosed herein can be employed in any pharmaceutically acceptable carrier whether or not the particular component is specifically listed with specific reference to a particular carrier type.

As has been described, bioorthogonal caged molecules are released upon contacting various releasing molecules such as tetrazines. In some examples, a general structure for such a releasing molecule is according to Structure 25:

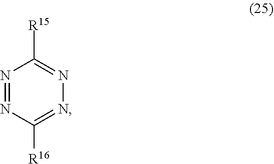

where R¹⁵ and R¹⁶ are independently selected from H, 2-pyridine, and Ph-CONH((CH₂)₂O)₃Me. In some examples, a general structure for such a releasing molecule is according to Structure 26:

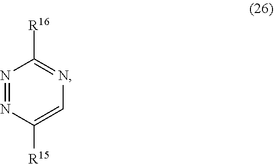

where R¹⁵ and R¹⁶ are independently selected from H, 2-pyridine, and Ph-CONH((CH₂)₂O)₃Me.

Any tetrazine molecule capable of generating an uncaging reaction with a caged molecule under bioorthogonal conditions is considered to be within the present scope. Nonlimiting tetrazine examples, however, can include 6-(6-(pyridin-2-yl)-1,4-dihydro-1,2,4,5-tetrazin-3-yl)pyridin-3-amine (Structure 27), 6-(6-(pyridin-2-yl)-1,2,4,5-tetrazin-3-yl)pyridin-3-amine (Structure 28), N-(methyl-PEG4)-6-(6-(pyridin-2-yl)-1,2,4,5-tetrazin-3-yl)pyridin (PEG-DPTz; Structure 29), 4-(6-methyl-1,2,4,5-tetrazin-3-yl)benzoic acid (Structure 30), 4-(6-methyl-1,2,4,5-tetrazin-3-yl)benzoic N-Hydroxysuccinimide (Structure 31), and 3,5-di(pyridine-2-yl)-1H-pyrazol-4-amine (DPPA; Structure 32), and the like. Each of these structures can be found in the example synthesis section below. It should be noted that the specific structures/molecules shown are merely exemplary and are not limiting.

In some examples, a method of reversibly modifying a target molecule can include removably coupling a bioorthogonal molecule as described herein to the target molecule and reacting the biorthogonal molecule with a releasing molecule to remove the bioorthogonal molecule from the target molecule.

In some examples of a method of reversibly modifying a target molecule, the bioorthogonal molecule is coupled to the target molecule via reaction of the target molecule with a reactive precursor of the bioorthogonal molecule.

In some examples of a method of reversibly modifying a target molecule, the bioorthogonal molecule is incorporated onto the target molecule during synthesis of the target molecule.

In some examples of a method of reversibly modifying a target molecule, coupling the biorthogonal molecule to the target molecule inactivates the target molecule.

In some examples of a method of reversibly modifying a target molecule, the biorthogonal molecule acts as a protecting group.

In some examples of a method of reversibly modifying a target molecule, the target molecule is a member of the group consisting of a polypeptide, a carbohydrate, a nucleic acid, a lipid, and combinations thereof.

In some examples, a method of administering a therapeutic agent to a subject can include administering a bioorthogonal molecule as described herein to the subject, the bioorthogonal molecule having the therapeutic agent releasably coupled thereto. The method also includes reacting the bioorthogonal molecule with a releasing molecule to separate the bioorthogonal molecule from the therapeutic agent.

In some examples of a method of administering a therapeutic agent to a subject, the bioorthogonal molecule is coupled to a carrier molecule.

In some examples of a method of administering a therapeutic agent to a subject, the therapeutic agent is released from the carrier molecule after reaction of the bioorthogonal molecule with the releasing molecule.

In some examples of a method of administering a therapeutic agent to a subject, the therapeutic agent is retained on the carrier molecule after reaction of the bioorthogonal molecule with the releasing molecule.

In some examples of a method of administering a therapeutic agent to a subject, the releasing molecule is coupled to a carrier molecule.

In some examples of a method of administering a therapeutic agent to a subject, the therapeutic agent is released from the bioorthogonal molecule after reaction of the bioorthogonal molecule with the releasing molecule coupled to the carrier molecule.

In some examples of a method of administering a therapeutic agent to a subject, the therapeutic agent is retained on the carrier molecule after reaction of the bioorthogonal molecule with the releasing molecule coupled to the carrier molecule.

In some examples, the bioorthogonal molecules can be used in methods of spatiotemporally controlled release of therapeutics or imaging agents in which compositions including a bioorthogonal molecule or a releasing molecule, at least one of which is linked to a carrier molecule, are co-administered simultaneously or sequentially with a time delay by any means of administration (e.g. topical, orally, intravenously, intramuscularly).

In some examples, the biooorthogonal molecules can be used in methods of spatio-temporally controlled release of therapeutics or imaging agents in which carrier molecules with attached bioorthogonal molecules are implanted at a specific location (e.g. hydrogel, stint, biomaterial) and administration of releasing molecules releases the therapeutic or imaging agent.

### Example Studies

### Investigation of Tz-Mediated ICPr/ICPrc-Group Removal

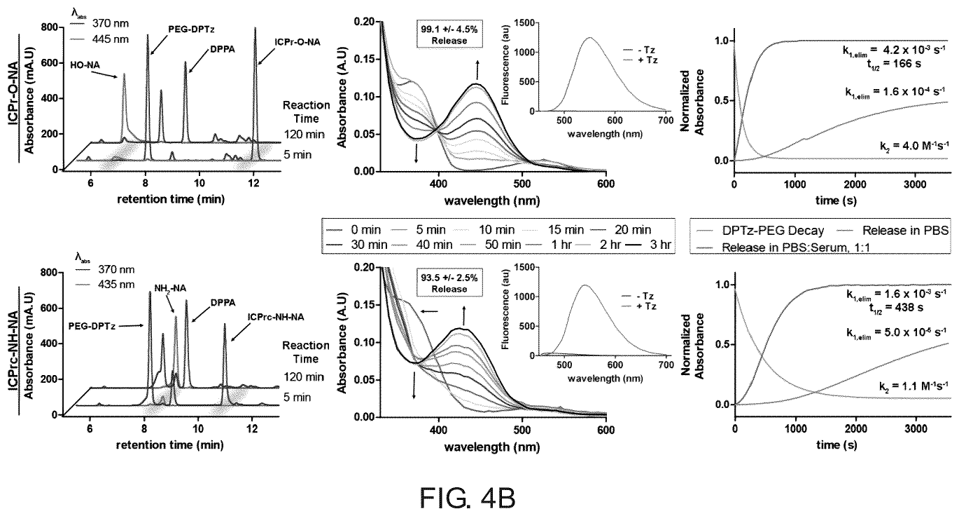

To investigate the Tz-mediated removal of ICPr/ICPrc-groups, probes were synthesized that report unmasking by ratiometric changes in absorbance and fluorescence spectra. 1,8-naphthalimides were modified on 4-OH/4-NH₂ functionalities with ICPr/ICPrc groups (3-isocyanopropyl derivative ICPr-O-NA and 3-isocyanopropyl-1-carbamoyl derivative ICPrc-NH-NA). A PEG4-group at the imine nitrogen endowed the probes with excellent water solubility. With these reporter molecules, it was confirmed that Tz elicits the traceless removal of ICPr/ICPrc groups from phenols and amines. Incubation of ICPr-O-NA and ICPrc-NH-NA (c=1 mM) with the water-soluble tetrazine PEG-DPTz (FIG. 4A; c=2 mM; T=37° C., PBS:DMSO (9:1, v/v)) led to the complete consumption of the masked dyes and the formation of the parental fluorophores as indicated by HPLC analysis (FIG. 4B, left column). FIG. 4A shows structures of caged 1,8-naphthalimide reporter probes and products of their reactions with tetrazine. More specifically, the structure on the left represents a caged PEG4-1,8-naphthalimide molecule having one of two R₂₀ caging groups from the bottom chart. The structure on the right represents an uncaged PEG4-1,8-naphthalimide molecule following the uncaging reaction. The uncaged PEG4-1,8-naphthalimide molecule includes an R₂₄ group from the bottom chart corresponding to the R₂₀ caging group pre-reaction caged molecule.

ICPr/ICPrc unmasking was further monitored by UV-Vis spectrophotometric analysis, as shown in FIG. 4B, center column. The introduced modifications caused a hypsochromic shift of the absorbance and emission bands of these fluorophores (FIG. 4A & 4B, center column). In case of ICPr-O-NA (c=1 mM), the absorbance band with a maximum at 370 nM disappeared rapidly in the presence of excess 3,6-di-2-pyridyl-1,2,4,5-tetrazine (DPTz; c=3 mM, T=37° C., PBS:DMSO (1:1)) concomitant with the appearance of the absorbance peak characteristic for HO-NA (λ_{abs,max}=445 nm) and an isobestic point at 395 nm. The Tz-mediated conversion of ICPrc-NH-NA to H₂N-NA, accompanied by an absorbance shift from 370 to 430 nm, provided a comparable result with the exception of a hypsochromic shift at early time points (FIG. 4B, center column), which indicated the formation of an intermediate with decreased electron-density on the amine. The formation of a cyclic 4-hydroxy-1,3-oxazinan-2-one at equilibrium with the 3-oxopropyl carbamate is a possible explanation for this observation as reported for related molecules. Measurement of unmasking yields based on the absorbance intensity of the product indicated near-quantitative release for the reaction of DPTz with ICPr-O-NA (99.1±4.5%) and ICPrc-NH-NA (93.5±2.5%). Liberation of the 1,8-naphthalimides further led to a strong fluorescence turn-on signal (ICPr-O-NA: 1210-fold; ICPrc-NH-NA: 76-fold; (FIG. 4B, center column inset). Removal of ICPr-groups also occurred with 7-hydroxycoumarin and resorufin (ICPr-res) fluorophores. Based on previous studies of β-eliminations from 3-oxopropyl substituents, it appears likely that ICPr/ICPrc chemistry can be used to mask diverse functional groups.

The kinetics of the Tz-induced release of phenols and amines from ICPr/ICPrc groups is shown in FIG. 4B, right column. Fitting the disappearance of PEG-DPTz (c=0.2 mM) absorbance in the presence of excess ICPr-O-NA or ICPrc-NH-NA (c=2 mM;T=37° C., PBS:DMSO (9:1)) to a pseudo-first order rate equation provided the bimolecular rate constants k₂(ICPr-O-NA)=4.0±0.2 M⁻¹s⁻¹ and k₂(ICPrc-NH-NA)=1.1±0.2 M⁻¹s⁻¹, respectively. Release of the fluorophores in PBS from the postulated 3-oxopropyl intermediate was delayed relative to the reaction of the ICPr/ICPrc-groups with PEG-DPTz (k_{1,elim}(HO-NA)=1.6×10⁻⁴ s⁻¹; k_{1,elim}(H₂N-NA)=5.00×10⁻⁵ s⁻¹; FIG. 4B, right column) in agreement with studies of cargo release from such groups. The aldehyde intermediate was detectable by NMR in DMSO-d₆:D₂O (9:1). To our delight, fluorophore release in diluted human serum (T=37° C., PBS:serum (1:1)) reached completion in few minutes because serum albumin can catalyze the β-elimination reaction. The apparent first-order rate constants for this step in serum were calculated as k_{1,elim}(ICPr-O-NA)=4.2×10⁻³ s⁻¹ and k_{1,elim}(ICPrc-NH-NA)=1.6×10⁻³ s⁻¹. In the absence of Tz, ICPr-O-NA and ICPr-NH-NA were completely stable in serum for at least three days. These outcomes demonstrate that stable ICPr/ICPrc-modifications can be rapidly and near-quantitatively removed from phenols and amines under physiological conditions.

FIG. 4B left column shows HPLC analysis of tetrazine-mediated uncaging of ICPr-O-NA and ICPr-NH-NA by PEG-DPTz (c(probe)=1 mM, c(Tz)=2 mM, PBS:DMSO (9:1), T=37° C.; DPPA: 3,5-di(pyrid-2-yl)-1H-pyrazol-4-amine). FIG. 4B center column shows time-dependent absorbance changes associated with the reaction of ICPr-O-NA and ICPr-NH-NA with DPTz (c(probe)=1 mM, c(Tz)=3 mM, PBS:DMSO (1:1), T=37° C.). FIG. 4B right column shows kinetics of tetrazine consumption and accumulation of products HO-NA and NH₂-NA (c(probe)=2 mM, c(Tz)=0.2 mM, PBS:DMSO (9:1), T=37° C.).

### Anticancer Agent Evaluation

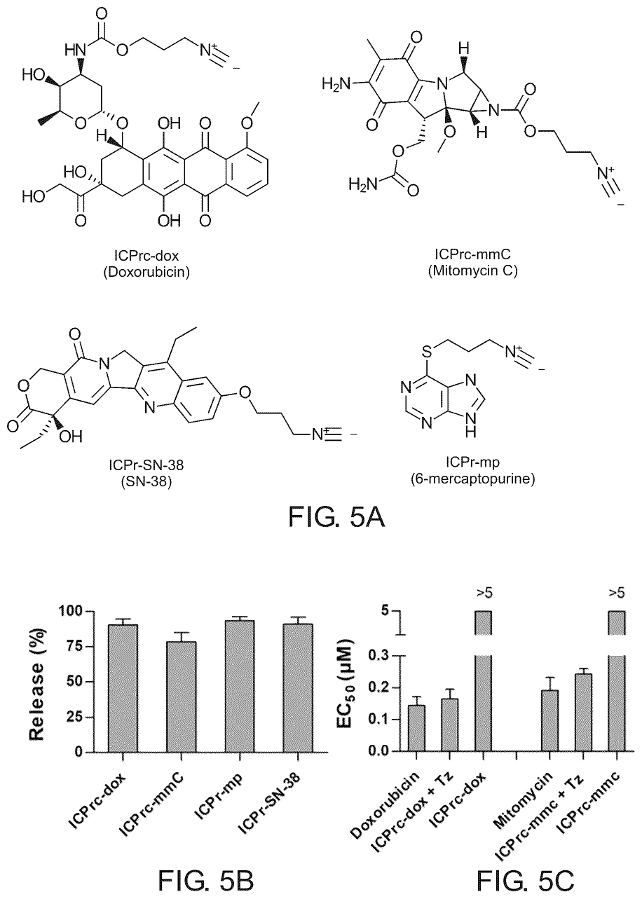

Tetrazine uncaging reactions were evaluated for caging and releasing various anticancer agents. As is shown in FIG. 5A, ICPr/ICPrc-prodrugs of doxorubicin (ICPrc-dox), mitomycin C (ICPr-mmc), mercaptopurine (ICPr-mp), and SN-38 (ICPr-SN-38) were synthesized by reacting the drugs with ICPr-tos or ICPr-nc. The prodrugs (c=1 mM) were incubated with DPTz (c=2 mM) in PBS:DMSO (1:1; T=37° C.; 2 mM GSH with exception of ICPr-SN-38) and the release was assessed by HPLC (t=4 h). Each of the drugs was released in high yields (FIG. 5B; ICPr-SN-38, 91±5%; ICPrc-dox, 91±4%; ICPr-mp, 94±3%; ICPrc-mmC=79±6%). To confirm that the released molecules were active, cytotoxicity experiments were performed with ICPr-dox and ICPrc-mmc (FIG. 5C).

Combinations of the prodrugs with excess PEG-DPTz (c=80 μM) elicited dose-dependent cytotoxicity in A549 adenocarcinoma cells. The potencies of the prodrugs combined with PEG-DPTz (EC₅₀ (ICPrc-dox)=0.165±0.035 μM; EC₅₀ (ICPrc-mmC)=0.244±0.017 μM) rivaled those of the free drugs (EC₅₀ (dox)=0.144±0.028 μM; EC₅₀ (mmC)=0.191±0.042 μM). In contrast, the prodrugs alone showed little toxicity in the tested concentration range (EC₅₀>5 μM). In conclusion, ICPr/ICPrc modifications can be used to generate tetrazine-responsive prodrugs for diverse bioactive compounds. FIG. 5B shows Release percentages for prodrugs (c(prodrug)=1 mM, c(DPTz)=2 mM, PBS:DMSO (1:1), T=37° C.). FIG. 4C shows EC₅₀ values in cytotoxicity studies (A549 cells) of prodrug alone, prodrug+Tz (c=80 μM), and parent drug.

### Cytotoxicity Assay

A549 lung cancer cells (ATCC, USA) were maintained in a humidified CO₂ (5%) incubator at 37° C. in RPMI (Thermo Fisher, USA) supplemented with 10% fetal bovine serum in the presence of 1% Penicillin-Streptomycin-Glutamine (Thermo Fisher, USA) and 0.2% Normocin (InvivoGen, USA).

The cells were plated in 96-well TC treated plates (PerkinElmer, USA) at a 6,000 cells/well density 24 h prior to the addition of the drugs. All drugs, prodrugs and PEG-DPTz in DMSO stock solution were serially diluted in pre-warmed culture medium at 37° C. For samples assessing the reaction-induced drug release, prodrugs were added to the cells first (100 μL final volume per well) in a series of final concentrations ranging from 0.005 to 10 μM followed by addition of PEG-DPTz (20, 40 and 80 μM).

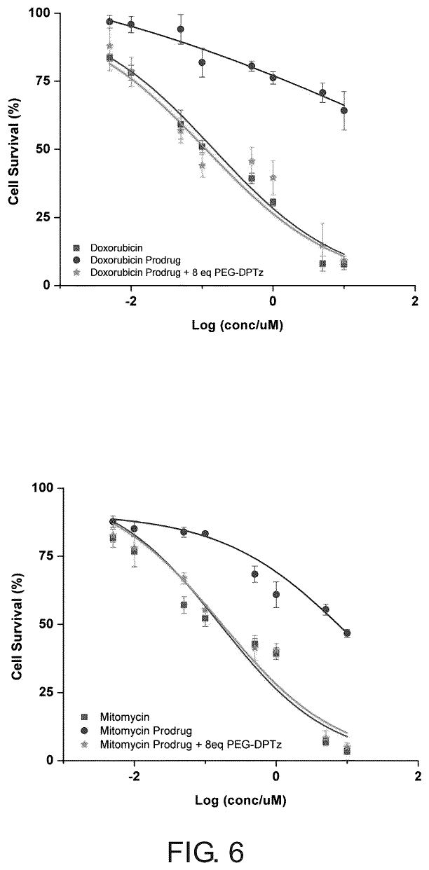

Doxorubicin and mitomycin C were used as the positive controls at the same concentrations. PEG-DPTz was also tested with same series of concentrations ranging from 0.05 to 100 μM, no obvious toxicity was observed. After 72 h incubation at 37° C., cell proliferation was assessed by a CellTiter-Glo©2.0 viability assay according to the manufacturer's instructions. After 15 min incubation at 25° C., the medium was gently measured with Envision 2104 Multilabel Reader (PerkinElmer, USA) to get the luminescent based on quantitation of the ATP present, an indicator of metabolically active cells. The proliferation assay was performed in quadruplet measurements (n=4). EC₅₀ values were derived from the normalized cell growth and Growth/Sigmoidal formula were fitted and generated with Origin 8.0. Results in A549 cells are also shown in FIG. 6. The top panel of FIG. 6 shows the measurement of cytotoxicity of reaction-activated doxorubicin in A549 lung adenocarcinoma cells and the bottom panel of FIG. 6 shows the measurement of cytotoxicity of reaction-activated mitomycin C in A549 lung adenocarcinoma cells. The results are expressed as the mean±standard deviation (n=4).

### In Vivo Activation in Zebrafish Embryo

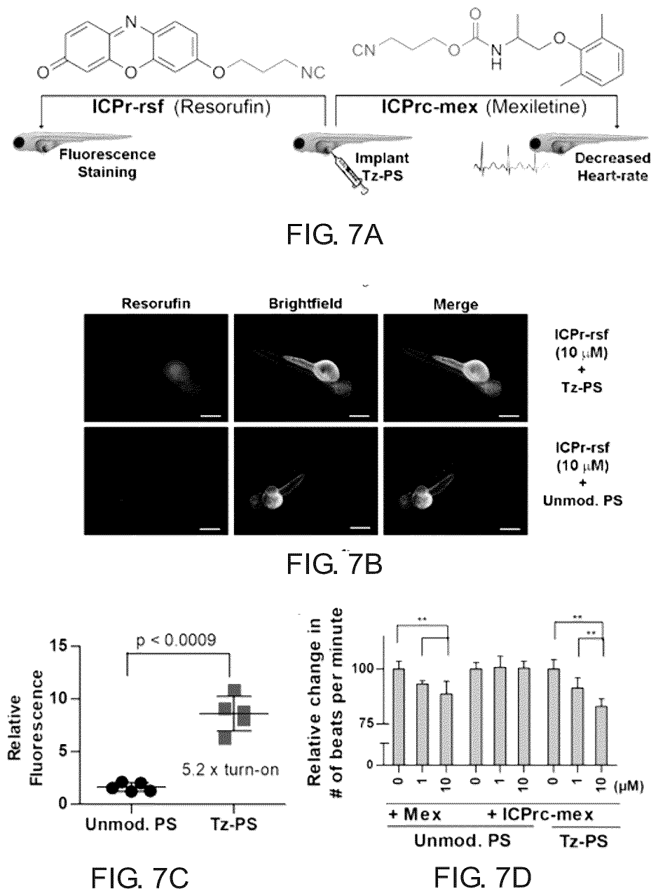

Zebrafish embryos were used as a model organism to evaluate the activity of uncaged payload molecules in vivo. FIG. 7A shows the structures of ICPr-modified resorufin (ICPr-rsf) and ICPrc-modified mexiletine (ICPrc-mex) along with an illustration of experiments involving the implantation into zebrafish of Tz-modified polystyrene bead (Tz-PS) followed by incubation with either ICPr-rsf for fluorescence imaging or ICPrc-mex leading to a decreased heart rate. For the fluorescence study, Tz-PSs were implanted into the yolk sacs of zebrafish embryos followed by incubation with ICPr-rsf (c=10 μM). Fish implanted with Tz-PS exhibited a significantly higher fluorescence staining when exposed to ICPr-rsf than controls with unmodified beads (5.2-fold, p-value=<0.0009, t=8 h; FIG. 7B & 7C). To corroborate that ICPr chemistry is compatible with living systems, the phenomenological effects of releasing an active drug inside zebrafish were also analyzed. An ICPr-prodrug of mexiletine (ICPrc-mex) was synthesized as shown in FIG. 7A. Mexiletine is a voltage-gated sodium channel blocker known to induce cardiac arrhythmia, and which has been reported to decrease heart rate. Incubation in ICPrc-mex containing medium (c=0, 1, 10 μM) caused a dose-dependent decrease in heart rate in fish with implanted Tz-PS similar to the effect observed for the free drug, whereas no changes were observed in control fish bearing unmodified beads (FIG. 7D). These experiments demonstrate that ICPr/ICPrc-derivatized molecules can be effectively unmasked in living organisms and liberate active compounds.

FIG. 7B shows detection of resorufin fluorescence upon tetrazine-mediated uncaging in zebrafish (scale bar=200 μm). FIG. 7C shows fluorescence increase in zebrafish with either Tz-PS or unmodified beads in when incubated with ICPr-rsf (c=10 μM; t=8 h). FIG. 7D shows the decrease in heart rate in zebrafish implanted with either Tz-PS or unmodified beads treated with ICPrc-mex (t=8 h; normalized to heart rate at t=0 h; **=p-value<0.005).

This evaluation has established the usefulness of ICPr/ICPrc moieties as masking groups that can be removed by reaction with Tz. In a series of steps, it has been demonstrated that the bimolecular reaction occurred rapidly, that release yields were near-quantitative, and that the chemistry was compatible with diverse molecules including reporter fluorophores and cytotoxic agents. Experiments in zebrafish models exemplified the utility of the chemistry for in vivo drug and probe release. One potential limitation of the ICPr/ICPRc groups is the delayed elimination of molecules from the 3-oxopropyl intermediate. In addition to the possibility of using albumin to accelerate the release, various simple structural modifications may be made to the design to afford near-instantaneous release. An intriguing aspect of ICPr/ICPRc groups is their structural compactness. Such moieties might be engineered into proteins for chemical control of activity while minimally disrupting their secondary structure or alternatively be used for designing prodrugs with little impact on their pharmacokinetics. The ease of synthesis will further make the outlined chemistry attractive for diverse applications in chemical biology and smart therapeutics.

### Example Procedures: Caging Molecule Synthesis

### Synthesis of 3-isocyanopropan-1-ol (ICPr-OH; Structure 04)

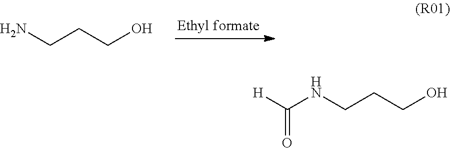 [SMILES: NCCCO ; O=CNCCCO]

Reaction R01 was performed by adding ethyl formate (11 mmol, 814 mg) portionwise to stirred 3-amino-propan-1-ol (10 mmol, 750 mg) over a period of 15 min. The solution was removed from the ice-bath and was heated at 50° C. for 2 h. Volatiles were removed by rotary evaporation to afford the desired product as a colorless oil in a near-quantitative yield (>95%) of N-(3-hydroxypropyl)-1-formamide. This compound decomposed upon storage and was immediately used in subsequent reactions, such as reaction R02. The NMR data is in agreement with spectra reported in the literature. ¹H NMR (400 MHz, CDCl₃) δ 8.16 (s, 1H), 3.68-3.65 (m, 2H), 3.46-3.41 (m, 2H), 1.74-1.68 (m, 2H).

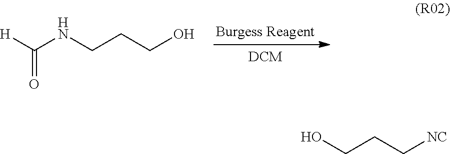

Reaction R02 was performed by adding Burgess Reagent (3.57 g, 15 mmol) portionwise over 10 min to a solution of N-(3-hydroxypropyl)-1-formamide (1.03 g, 10 mmol) in dry CH₂Cl₂ (25 mL). The solution was stirred at room temperature under nitrogen atmosphere for 12 h. The mixture was diluted with CH₂Cl₂ (50 mL), concentrated under reduced pressure, and purified by column chromatography (Hexane:EA=3:1, v/v; R_{f}=0.35) to afford the 3-isocyanopropan-1-ol (ICPr-OH; Structure 04) as a yellow solid in a yield of 206 mg (25%). ¹H NMR (400 MHz, CDCl₃) δ 3.61 (t, J=6.0 Hz, 2H), 3.45-3.42 (m, 2H), 1.79-1.75 (m, 2H). ¹³C NMR (100 MHz, CDCl₃) δ 154.2, 57.7, 38.4, 38.3, 31.5.

### Synthesis of 3-isocyano-1-tosylpropane (ICPr-tos; Structure 05)

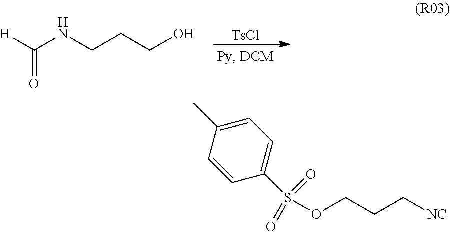

Reaction R03 was performed by adding a solution of tosyl chloride (7.4 g, 38.8 mmol) in dry DCM (20 mL) dropwise over 30 minutes to a stirred solution of N-(3-hydroxypropyl)-1-formamide (1 g, 9.70 mmol) in dry pyridine (20 mL). The solution was stirred at 0° C. under a nitrogen atmosphere for 4 hours. The reaction was quenched with ice cold water and extracted with a mixture of diethyl ether:hexane (3:1, 2×30 mL). The organic layer was washed with water (3×50 mL) and brine (1×50 mL), dried over Mg₂SO₄, filtrated, and concentrated under reduced pressure. The residue was dissolved in a 1:1 DCM, hexane mixture and purified by column chromatography (DCM; R_{f}=0.5) to afford the 3-isocyano-1-tosylpropane (ICPr-tos; Structure 05) as an oil in a yield of 1.5 g (65%).

¹H NMR (400 MHz, CDCl₃) δ 7.79 (d, J=8.2 Hz, 2H), 7.37 (d, J=8.2 Hz, 2H), 4.16 (t, J=5.8 Hz, 2H), 3.50-3.48 (m, 2H), 2.45 (s, 3H), 2.07-1.95 (m, 2H). ¹³C NMR (100 MHz, CDCl₃) δ 160.5, 157.5, 145.3, 132.4, 130.1, 127.9, 77.4, 77.1, 76.7, 65.9, 59.8, 37.8, 28.6, 21.7.

### Synthesis of 3-isocyanopropyl-1-(4-nitrophenyl)carbonate (ICPr-nc; Structure 06)

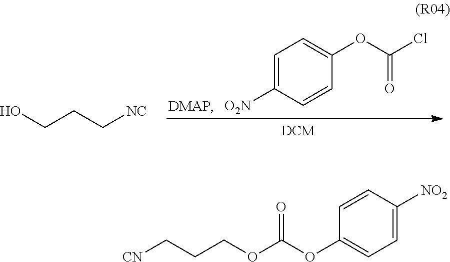

Reaction R04 was performed by adding 4-(dimethylamino)pyridine (122 mg, 1 mmol) and nitrophenyl chloroformate (142 mg, 0.7 mmol) to a solution of 3-isocyanopropan-1-ol (41 mg, 0.48 mmol) in dry CH₂Cl₂ (6 mL) at 0° C. The solution was kept at 25° C. overnight (12 h). The mixture was quenched with ice and extracted with CH₂Cl₂ (2×20 mL). The combined organic layers were washed with water (3×50 mL) and brine (3×50 mL) until no more yellow color was observed in the organic phase, dried over Na₂SO₄ and concentrated to afford 3-isocyanopropyl-1-(4-nitrophenyl)carbonate (ICPr-nc; Structure 06) as a light yellow solid in a yield of 85 mg (64%). This compound decomposed gradually upon storage and was immediately used in subsequent reactions. ¹H NMR (400 MHz, CDCl₃) δ 8.29 (d, J=9.2 Hz, 2H), 7.39 (d, J=9.2 Hz, 2H), 4.46 (t, J=6.0 Hz, 2H), 3.62 (t, J=6.4 Hz, 2H), 2.17-2.15 (m, 2H).

### Synthesis of 3-isocyanide-2-phenylpropan-1-ol (Structure 07)

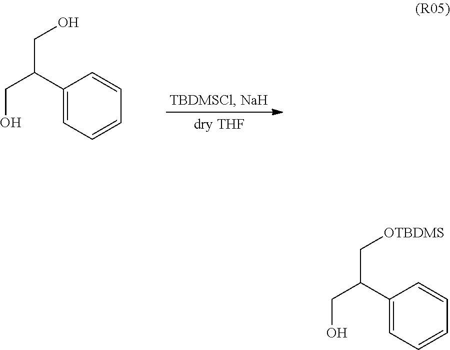 [SMILES: OCC(CO)c1ccccc1 ; CC(C)(C)[Si](C)(C)OCC(CO)c1ccccc1]

Reaction R05 was performed by adding a solution of 2-phenylpropane-1,3-diol (5 g, 33 mmol) in THE (25 mL) dropwise to a stirred and ice cooled suspension of NaH (1.5 g, 36 mmol) in THE (15 mL). The ice bath was removed, and reaction was warmed up to room temperature for 12 h. The mixture was quenched with ice, diluted with EtOAc (300 mL) and washed with brine (2×150 mL). The separated organic layer was dried with Na₂SO₄, and concentrated under reduced pressure, purified by column chromatography (100% hexane) to afford 3-((tert-butyldimethylsilyl)oxy)-2-phenylpropan-1-ol as yellow solid in a yield of 5 g (59%; R_{f}=0.8 in hexane. EtOAc=100:1, v/v). ¹H NMR (400 MHz, CDCl₃) δ 7.33-7.30 (m, 2H), 7.24 (brs, 1H), 7.22-7.20 (m, 2H), 4.13-4.05 (m, 2H), 3.93-3.87 (m, 2H), 3.12-3.05 (m, 1H), 1.26 (t, J=7.2 Hz, 2H), 0.90 (s, 9H), 0.06 (s, 6H). The ¹H NMR data agreed with the reported spectrum of this compound.

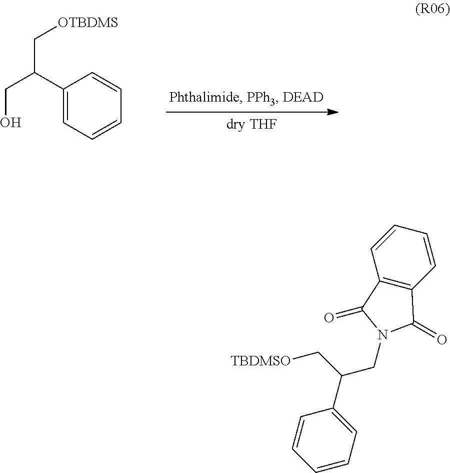 [SMILES: CC(C)(C)[Si](C)(C)OCC(CO)c1ccccc1 ; CC(C)(C)[Si](C)(C)OCC(CN1C(=O)c2ccccc2C1=O)c1ccccc1]

Reaction R06 was performed by sequentially adding PPh₃ (6.3 g, 24 mmol) and Pht (2.8 g, 19.2 mmol) to a stirred and ice cooled solution of 3-((tert-butyldimethylsilyl)oxy)-2-phenylpropan-1-ol (4.4 g, 16 mmol) in THE (60 mL). To such mixture, diethyl azodicarboxylate (3.34 g, 19.2 mmol) in THE (15 mL) was then added dropwise. The ice bath was removed, and reaction was warmed up to room temperature for 12 h. The mixture was quenched with ice, diluted with Et₂O (200 mL) and washed with sat NaHCO₃(2×150 mL) and brine (2×150 mL). The separated organic layer was dried with Na₂SO₄, and concentrated under reduced pressure, purified by column chromatography (hexane:EtOAc=10:1, v/v) to afford 2-(3-((tert-butyldimethylsilyl)oxy)-2-phenylpropyl)isoindoline-1,3-dione as yellow oil in a yield of 3.2 g (50%; R_{f}=0.6 in hexane:EtOAc=5:1, v/v). ¹H NMR (400 MHz, CDCl₃) δ 7.78-7.76 (m, 2H), 7.66-7.64 (m, 2H), 7.25-7.18 (m, 5H), 4.11-4.00 (m, 2H), 3.85-3.81 (m, 2H), 3.49-3.44 (m, 1H), 1.26 (t, J=7.2 Hz, 2H), 0.81 (s, 9H), 0.09 (s, 6H). The ¹H NMR data agreed with the reported spectrum of this compound.

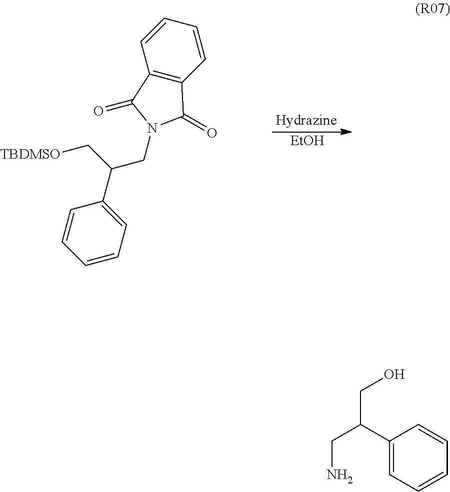 [SMILES: "EtOH" ; CC(C)(C)[Si](C)(C)OCC(CN1C(=O)c2ccccc2C1=O)c1ccccc1 ; NCC(CO)c1ccccc1]

Reaction R07 was performed by adding hydrazine hydrate (2 g, 40 mmol) to a stirred and ice cooled solution of 2-(3-((tert-butyldimethylsilyl)oxy)-2-phenylpropyl)isoindoline-1,3-dione (1.6 g, 4 mmol) in EtOH (40 mL) and the reaction was refluxed for 2 h. The mixture was quenched with ice, diluted with Et₂O (200 mL) and washed with sat NaHCO₃(2×150 mL). The aqueous layer was extracted with Et₂O (100 mL) and combined organic layer was washed with 1 M HCl solution. The resulting acidic solution was then treated with 10 M NaOH until pH 11 and washed with EtOAc (200 mL). The combined organic layer was dried with Na₂SO₄, and concentrated under reduced pressure to give 3-amino-2-phenylpropan-1-ol as yellow oil in a yield of 185 mg (30%; R_{f}=0.1 in EtOAc:MeOH=5:1, v/v). ¹H NMR (400 MHz, CDCl₃) δ 7.36-7.30 (m, 3H), 7.24-7.22 (m, 2H), 4.10-4.04 (m, 2H), 3.99 (t, J=7.6 Hz, 1H), 3.54-3.49 (m, 1H), 3.32-3.26 (m, 1H). HRMS (ESI): calcd. for C₉H₁₃NO [M+H]⁺ 152.1070, found 152.1095. The ¹H NMR data agreed with the reported spectrum of this compound.

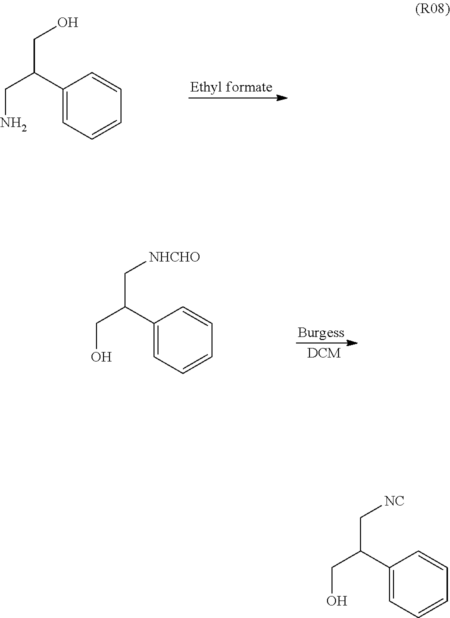

Reaction R08 was performed by adding ethyl formate (850 mg, 12 mmol) portionwise to a stirred and ice cooled solution of 3-amino-2-phenylpropan-1-ol (185 mg, 1.2 mmol). The solution was removed from the ice-bath and was heated at 50° C. for 2 h. Excess ethyl formate was removed by rotary evaporation to afford the desired product as a colorless oil in a near-quantitative yield (>95%). This compound decomposed upon storage and was immediately used in the next step. Burgess Reagent (350 mg, 1.5 mmol) was added portionwise over 10 min to a solution of the aforementioned intermediate in dry CH₂Cl₂ (2.5 mL). The solution was stirred at room temperature under nitrogen atmosphere for 2 h. The mixture was diluted with CH₂Cl₂ (50 mL), washed with brine (50 mL), concentrated under reduced pressure, and purified by column chromatography (Hexane:EA=3:1, v/v) to afford 3-isocyanide-2-phenylpropan-1-ol (Structure 07) as a yellow solid in a yield of 106 mg (55%; R_{f}=0.3 in EtOAc:hexane=1:3, v/v). ¹H NMR (400 MHz, CDCl₃) δ 7.39-7.31 (m, 3H), 7.24-7.22 (m, 2H), 3.92-3.77 (m, 2H), 3.73-3.68 (m, 2H), 3.12 (brs, 1H). ¹³C NMR (100 MHz, CDCl₃) δ 156.8, 156.7, 137.8, 128.9, 127.8, 127.7, 63.1, 46.9, 43.5, 43.5, 43.4. HRMS (ESI): calcd. for C₁₀H₁₁NO [M+H]⁺ 162.0913, found 162.0929.

### Synthesis of 3-isocyano-2-phenyl-1-tosylpropane (Structure 08)

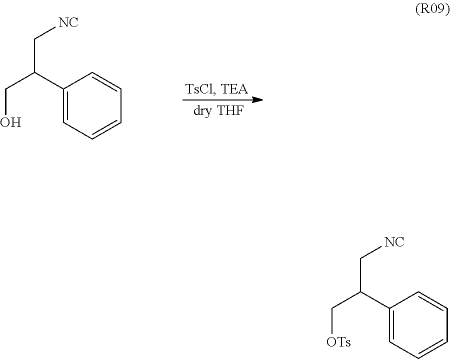

Reaction R09 was performed by adding a solution of tosyl chloride (114 mg, 0.6 mmol) and TEA (60 mg, 0.6 mmol) in dry THF (1 mL) to a stirred solution of 3-isocyanide-2-phenylpropan-1-ol (65 mg, 0.4 mmol) in dry THF (1 mL) dropwise over 30 minutes. The solution was stirred at 0° C. under a nitrogen atmosphere for 4 hours. The reaction was diluted with DCM (20 mL) and washed with brine (2×30 mL). The organic layer was dried over Na₂SO₄, concentrated under reduced pressure and purified by column chromatography (100% DCM) to afford 3-isocyano-2-phenyl-1-tosylpropane (Structure 08) as an oil in a yield of 75 mg (R_{f}=0.3 in DCM:hexane=10:1, v/v). ¹H NMR (400 MHz, CDCl₃) δ 7.74 (d, J=8.0 Hz, 2H), 7.35-7.32 (m, 5H), 7.16 (d, J=8.0 Hz, 2H), 4.28-4.25 (m, 2H), 3.73 (t, J=4.4 Hz, 2H), 3.29 (t, J=6.4 Hz, 1H), 2.45 (s, 3H). ¹³C NMR (100 MHz, CDCl₃) δ 145.2, 135.6, 132.2, 130.0, 129.0, 128.4, 127.9, 127.6, 69.2, 43.9, 21.6, 14.1. HRMS (ESI): calcd. for C₁₇H₁₇NO₃S [M+H]⁺ 316.1002, found 316.1005.

### Synthesis of 3-isocyano-1-phenylpropan-1-ol (Structure 09)

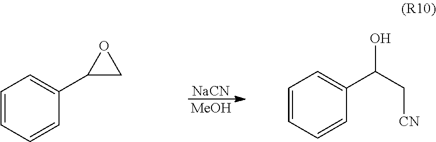

Reaction R10 was performed by adding a solution of sodium cyanide (2 g, 100 mmol) in water (20 mL) to a solution of styrene oxide (4 mL, 35 mmol) in MeOH (100 mL). The reaction was allowed to stir for 12 h. The mixture was then quenched with water (80 mL) and 2 N HCl solution (100 mL). CAUTION: evolution of hydrogen cyanide gas! The reaction was diluted with DCM (400 mL) and washed with brine (2×200 mL). The organic layer was dried over Na₂SO₄, concentrated under reduced pressure and purified by column chromatography (hexane:EA=6:1, v/v) to afford 2-cyano-1-phenylethanol as an oil in a yield of 3 g. (62%, R_{f}=0.2 in hexane:EA=6:1, v/v). ¹H NMR (400 MHz, CDCl₃) δ 7.41-7.36 (m, 5H), 5.05 (t, J=6.4 Hz, 1H), 2.78 (d, J=6.4 Hz, 2H). The ¹H NMR data agreed with the reported spectrum of this compound.

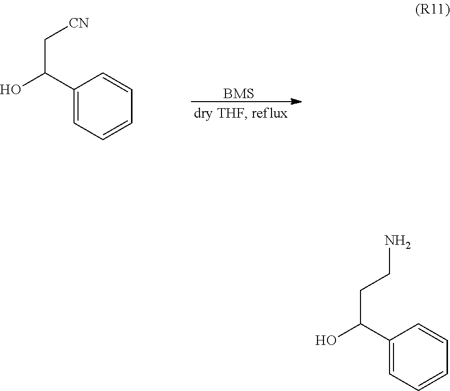

Reaction R11 was performed by adding a solution of borane-dimethyl sulfide complex (2.2 mL, 23 mmol) in dry THF(10 mL) dropwise to a solution of 2-cyano-1-phenylethanol (3 g, 21 mmol) in dry THF (10 mL) at 0° C. with stirring under N₂ gas. The mixture was reflux for 8 h. The mixture was quenched by ice water, diluted with EtOAc (200 mL) and washed with brine (200 mL). The combined organic layer was dried over Na₂SO₄, concentrated under reduced pressure and purified by column chromatography (EA:MeOH=1:1, v/v) to afford a colorless oil of 3-amino-1-phenylpropan-1-ol in a yield of 600 mg (20%, R_{f}=0.15 in EA:MeOH=1:1, v/v). ¹H NMR (400 MHz, CDCl₃) δ 7.39-7.23 (m, 5H), 4.97 (d, J=8.4 Hz, 1H), 2.78 (brs, 2H), 1.89-1.74 (m, 2H). HRMS (ESI): calcd. for C₉H₁₃NO [M+H]⁺ 152.1070, found 152.1062. The ¹H NMR data agreed with the reported spectrum of this compound.

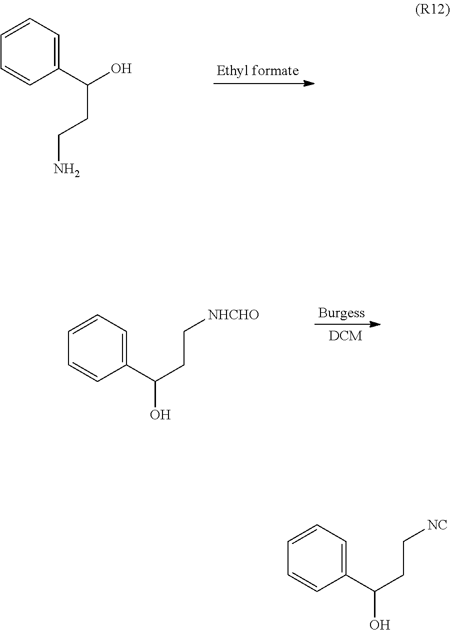

Reaction R12 was performed by adding ethyl formate (850 mg, 12 mmol) portionwise to a stirred and ice cooled solution of 3-amino-1-phenylpropan-1-ol (185 mg, 1.2 mmol). The solution was removed from the ice-bath and was heated at 50° C. for 2 h. Excess ethyl formate was removed by rotary evaporation to afford the desired product as a colorless oil in a near-quantitative yield (>95%). This compound decomposed upon storage and was immediately used in the next step. Burgess Reagent (350 mg, 1.5 mmol) was added portionwise over 10 min to a solution of the aforementioned intermediate in dry CH₂Cl₂ (2.5 mL). The solution was stirred at room temperature under nitrogen atmosphere for 2 h. The mixture was diluted with CH₂Cl₂ (50 mL), washed with brine (50 mL), concentrated under reduced pressure, and purified by column chromatography (Hexane:EA=3:1, v/v) to afford 3-isocyanide-1-phenylpropan-1-ol (Structure 09) as a colorless oil in a yield of 220 mg (35%, R_{f}=0.35 in EA:hexane=1:3, v/v). ¹H NMR (400 MHz, CDCl₃) δ 7.38-7.31 (m, 5H), 4.90-4.86 (m, 1H), 3.67-3.59 (m, 1H), 3.47-3.39 (m, 1H), 2.08-2.03 (m, 1H). ¹³C NMR (100 MHz, CDCl₃) δ 156.0, 156.0, 155.9, 143.1, 128.7, 128.3, 128.1, 125.6, 70.6, 38.6, 38.5, 38.4, 37.9, 29.7. HRMS (ESI): calcd. for C₁₀H₁₁NO [M+H]⁺ 162.0913, found 162.0905.

### Example Procedures: Caging Molecule Synthesis

### Synthesis of N-(methyl-PEG4)-4-(3-isocyanopropyl-carbamoyl)amino-1,8-naphthalimide (ICPrc-NH-NA; Structure 12)

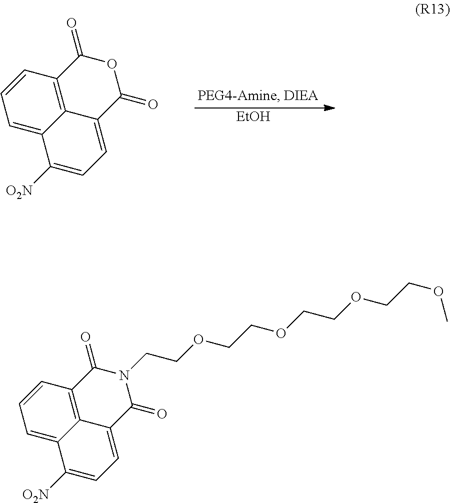 [SMILES: "EtOH" ; O=C1OC(=O)c2ccc([N+](=O)[O-])c3cccc1c23 ; COCCOCCOCCOCCN1C(=O)c2cccc3c([N+](=O)[O-])ccc(c23)C1=O]

Reaction R13 was performed by adding PEG4-Amine (228 mg, 1.1 mmol) to a solution of 4-nitro-1,8-naphthalic anhydride (243 mg, 1 mmol) and DIEA (162 mg, 1.25 mmol) in EtOH (10 mL). The solution was stirred at 80° C. for 2 h. Then the mixture was concentrated under reduced pressure and purified by column chromatography (Hexane:EA=1:2, v/v; R_{f}=0.45) to afford N-(methyl-PEG4)-4-nitro-1,8-naphthalimide as yellow oil in a yield of 227 mg (55%). ¹H NMR (400 MHz, CDCl₃) δ 8.60 (dd, J₁=1.2 Hz, J₂=8.8 Hz, 1H), 8.55 (t, J=8.4 Hz, 2H), 8.36 (d, J=8.0 Hz, 1H), 7.93 (q, J=7.2 Hz, 1H), 4.35 (t, J=6.0 Hz, 2H), 3.80 (t, J=6.0 Hz, 2H), 3.67-3.65 (m, 2H), 3.59-3.57 (m, 2H), 3.54-3.50 (m, 4H), 3.48-3.43 (m, 4H), 3.30 (s, 3H). ¹³C NMR (100 MHz, CDCl₃) δ 163.2, 162.4, 149.5, 131.6, 129.5, 128.6, 128.5, 126.4, 123.6, 123.1, 122.7, 71.5, 70.1, 70.0, 69.9, 69.8, 67.3, 57.3, 39.1.

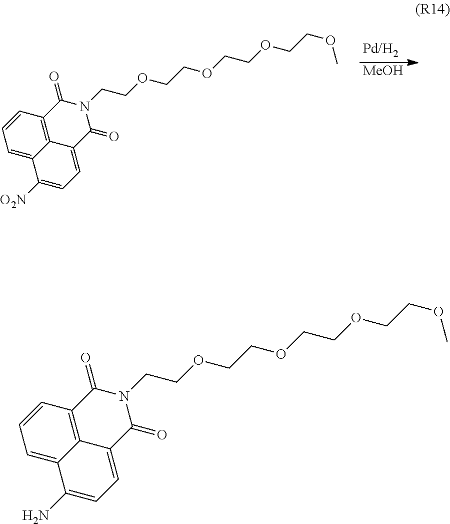 [SMILES: "MeOH" ; COCCOCCOCCOCCN1C(=O)c2cccc3c([N+](=O)[O-])ccc(c23)C1=O ; COCCOCCOCCOCCN1C(=O)c2cccc3c(N)ccc(c23)C1=O]

Reaction R14 was performed by adding Pd/C (5 wt %, 100 mg) to a solution of N-(methyl-PEG4)-4-nitro-1,8-naphthalimide (216 mg, 0.5 mmol) in EtOH (10 mL). The solution was stirred at room temperature under nitrogen atmosphere for 2 h. Silica gel was added and the mixture was evaporated under reduced pressure and the residue was purified by column chromatography (Hexane:EA=1:1, v/v; R_{f}=0.50) to afford N-(methyl-PEG4)-4-amino-1,8-naphthalimide (NH₂-NA) as yellow oil in a yield of 128 mg (65%). ¹H NMR (400 MHz, CDCl₃) δ 8.50-8.46 (m, 2H), 8.25 (d, J=8.0 Hz, 1H), 7.63-7.59 (m, 1H), 6.85 (d, J=8.0 Hz, 1H), 4.36 (t, J=6.4 Hz, 2H), 3.78 (t, J=6.4 Hz, 2H), 3.67-3.65 (m, 2H), 3.59-3.57 (m, 2H), 3.54-3.49 (m, 4H), 3.48-3.45 (m, 4H), 3.31 (s, 3H). ¹³C NMR (100 MHz, CDCl₃) δ 164.9, 164.3, 153.1, 134.1, 131.2, 130.1, 128.8, 123.8, 121.9, 119.7, 108.2, 108.1, 71.4, 70.1, 70.0, 69.9, 69.8, 67.7, 57.6, 39.0.

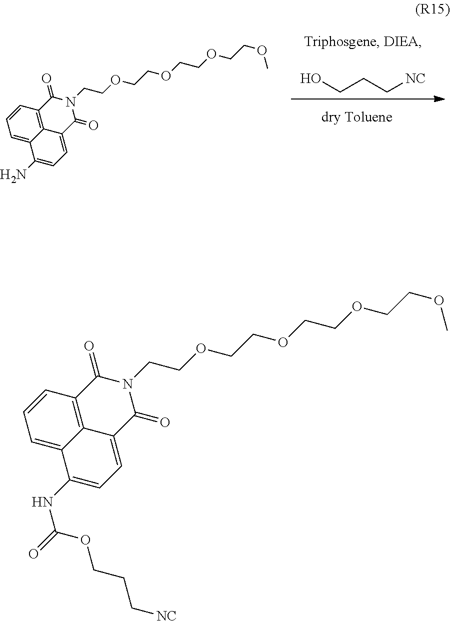

Reaction R15 was performed by adding dropwise a solution of triphosgene (78 mg, 0.26 mmol) in dry toluene (1 mL) to a mixture of N-(methyl-PEG4)-4-amino-1,8-naphthalimide (96 mg, 0.24 mmol) and DIEA (88 mg, 0.72 mmol) in dry toluene (5 mL). The solution was heated to reflux for 3 h. After cooling to room temperature, the mixture was added another solution of 3-isocyanopropan-1-ol (41 mg, 0.48 mmol) in dry CH₂Cl₂ (6 mL) and the reaction was stirred at room temperature for 12 h. Then the mixture was concentrated under reduced pressure and purified by column chromatography (Hexane:EA=2:3, v/v; R_{f}=0.35) to afford N-(methyl-PEG4)-4-(3-isocyanopropyl-carbamoyl)amino-1,8-naphthalimide (ICPrc-NH-NA; Structure 12) as yellow oil in a yield of 41 mg (35%).

¹H NMR (400 MHz, CDCl₃) δ 8.40-8.37 (m, 2H), 8.21 (dd, J₁=1.2 Hz, J₂=8.4 Hz, 1H), 8.18-8.14 (m, 1H), 7.60 (t, J=8.0 Hz, 1H), 4.44 (t, J=6.0 Hz, 2H), 4.37 (t, J=6.4 Hz, 2H), 3.85 (t, J=6.4 Hz, 2H), 3.74-3.72 (m, 2H), 3.68-3.64 (m, 4H), 3.58-3.55 (m, 2H), 3.48-3.45 (m, 4H), 3.43-3.38 (m, 2H), 3.28 (s, 3H), 2.15 (brs, 2H). ¹³C NMR (100 MHz, CDCl₃) δ 164.1, 163.6, 156.9, 153.1, 153.0, 139.3, 139.2, 132.4, 131.1, 128.5, 126.7, 126.3, 122.9, 122.8, 122.7, 117.3, 116.9, 116.8, 71.7, 70.58, 70.52, 70.46, 70.38, 70.31, 70.2, 68.1, 62.0, 58.9, 58.9, 39.2, 38.7, 38.6, 28.6.

### Synthesis of N-(methyl-PEG4)-4-(3-isocyanopropyl-1-oxy)-1,8-naphthalimide (ICPr-O-NA; Structure 13)

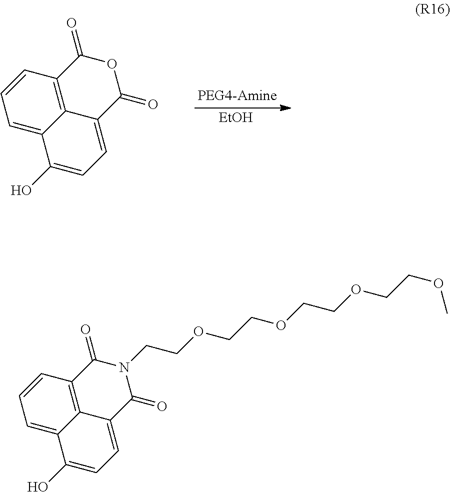 [SMILES: "EtOH" ; O=C1OC(=O)c2ccc(O)c3cccc1c23 ; COCCOCCOCCOCCN1C(=O)c2cccc3c(O)ccc(c23)C1=O]

Reaction R16 was performed by adding PEG4-Amine (147 mg, 0.71 mmol) to a solution of 4-hydroxy-1,8-naphthalic anhydride (100 mg, 0.47 mmol) in EtOH (7 mL). The solution was stirred at 80° C. overnight (12 h). The mixture was concentrated under reduced pressure and the residue was purified by column chromatography (DCM:MeOH=20:1, v/v; R_{f}=0.2) to afford N-(methyl-PEG4)-4-hydroxy-1,8-naphthalimide (HO-NA; 7) as yellow oil in a yield of 75 mg (40%). ¹H NMR (400 MHz, MeOD-d₄) δ 8.62 (d, J=7.8 Hz, 1H), 8.51 (d, J=6.6 Hz, 1H), 8.37 (d, J=8.4 Hz, 1H), 7.65 (t, J=7.8 Hz, 1H), 6.94 (d, J=8.4 Hz, 1H), 4.37 (t, J=6.2 Hz, 2H), 3.78 (d, J=6.0 Hz, 2H), 3.68-3.61 (m, 3H), 3.58 (d, J=4.8 Hz, 2H), 3.54-3.47 (m, 4H), 3.47-3.40 (m, 4H). ¹³C NMR (125 MHz, MeOD-d₄) δ 164.2, 163.4, 133.4, 130.2, 130.0, 129.0, 123.2, 120.7, 109.9, 109.8, 70.5, 69.2, 69.1, 69.1, 69.1, 69.0, 69.0, 66.9, 56.8, 52.5, 37.8.

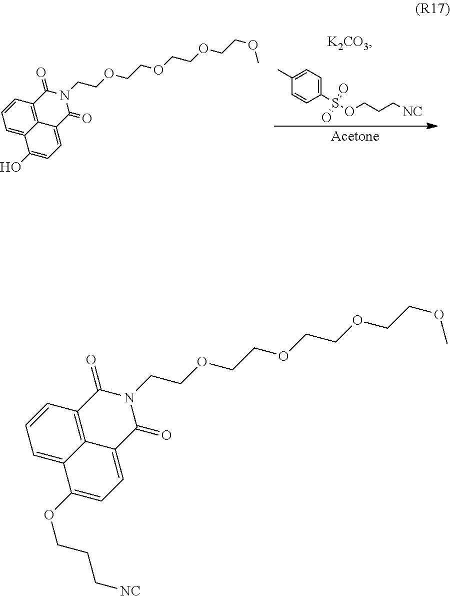

Reaction R17 was performed by adding anhydrous K₂CO₃ (26 mg, 0.19 mmol) to a mixture of N-(methyl-PEG4)-4-hydroxy-1,8-naphthalimide (50 mg, 0.12 mmol) in acetone (1 mL) and the mixture was allowed to stir at room temperature for 15 minutes. 3-isocyano-1-tosylpropane (89 mg, 0.37 mmol) dissolved in acetone (0.2 mL) was added dropwise and the reaction mixture was allowed to stir overnight at 50° C. (16 h). The solution was cooled to room temperature, solid residues was removed by filtration, and the filtrate concentrated under reduced pressure. The residue was purified by preparative TLC (DCM:MeOH=50:1, v/v, R_{f}=0.25) to afford N-(methyl-PEG4)-4-(3-isocyanopropyl-1-oxy)-1,8-naphthalimide (ICPr-O-NA; Structure 13) as an orange oil in a yield of 25 mg (44%).

¹H NMR (400 MHz, MeOD-d₄) δ 8.50 (d, J=8.2 Hz, 1H), 8.42 (d, J=6.8 Hz, 1H), 8.37 (d, J=8.2 Hz, 1H), 7.67 (t, J=7.8 Hz, 1H), 7.16 (d, J=8.2 Hz, 1H), 4.43 (t, J=5.8 Hz, 2H), 4.32 (t, J=6.0 Hz, 2H), 3.86 (t, J=6.2 Hz, 2H), 3.77 (t, J=6.2 Hz, 2H), 3.71-3.60 (m, 3H), 3.58 (dd, J=5.8, 3.2 Hz, 3H), 3.55-3.47 (m, 4H), 3.44 (dd, J=9.2, 4.2 Hz, 4H), 2.36 (s, 2H). ¹³C NMR (100 MHz, MeOD-d₄) δ 164.3, 163.7, 159.8, 133.2, 131.1, 128.9, 128.4, 125.8, 123.1, 121.8, 114.5, 106.0, 71.4, 70.1, 70.0, 69.9, 69.8, 67.5, 65.4, 57.6, 48.2, 48.0, 47.8, 47.6, 47.4, 47.1, 46.9, 38.7, 38.3, 38.3, 38.2, 28.5.

### Synthesis of N-(methyl-PEG4)-4-(3-isocyanopropyl-2-phenylcarbamoyl)amino-1,8-naphthalimide (ICPrc-2-Ph-NH-NA; Structure 14)

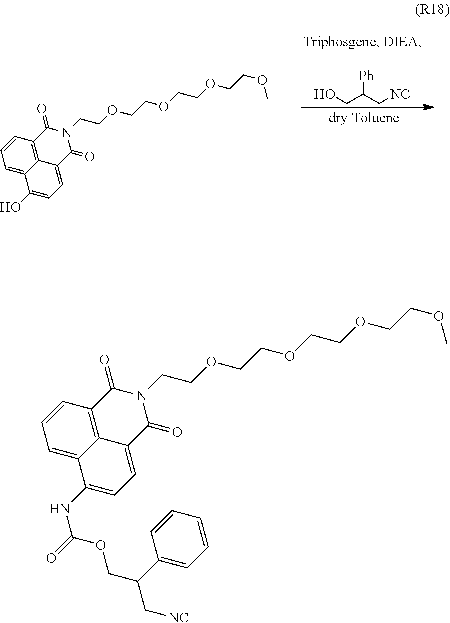

Reaction R18 was performed by adding dropwise a solution of triphosgene (39 mg, 0.13 mmol) in dry toluene (1 mL) to a mixture of N-(methyl-PEG4)-4-amino-1,8-naphthalimide (48 mg, 0.12 mmol) and DIEA (44 mg, 0.36 mmol) in dry toluene (2.5 mL). The solution was heated to reflux for 3 h. After cooling to room temperature, a solution of 3-isocyanide-2-phenylpropan-1-ol (39 mg, 0.24 mmol) in dry CH₂Cl₂ (3 mL) was added to the mixture and the reaction was stirred at room temperature for 12 h. Then the mixture was concentrated under reduced pressure and purified by column chromatography (DCM: MeOH=20:1, v/v) to afford N-(methyl-PEG4)-4-(3-isocyanopropyl-2-phenylcarbamoyl)amino-1,8-naphthalimide (ICPrc-2-Ph-NH-NA; Structure 14) as yellow oil in a yield of 21 mg (32%, R_{f}=0.25 in DCM:MeOH=20:1, v/v).

¹H NMR (400 MHz, MeOD-d₄) δ 8.54 (d, J=7.6 Hz, 1H), 8.48 (t, J=7.2 Hz, 2H), 8.14 (d, J=7.6 Hz, 1H), 7.80-7.76 (m, 1H), 7.41-7.33 (m, 5H), 4.56-4.53 (m, 2H), 4.37 (t, J=6.0 Hz, 2H), 4.03-3.91 (m, 2H), 3.79 (t, J=6.0 Hz, 2H), 3.66-3.64 (m, 2H), 3.58-3.56 (m, 2H), 3.51-3.46 (m, 5H), 3.43-3.41 (m, 4H), 3.29 (s, 3H). ¹³C NMR (100 MHz, MeOD-d₄) δ 163.8, 163.3, 154.0, 140.9, 138.2, 132.0, 131.3, 129.73, 129.0, 128.8, 128.7, 128.5, 128.5, 128.0, 126.7, 124.3, 122.5, 118.7, 118.4, 117.5, 71.6, 70.2, 70.1, 70.0, 69.9, 67.3, 65.7, 58.4, 44.0. HRMS (ESI): calcd. for C₃₂H₃₅N₃O₈ [M+Na]⁺ 612.2316, found 612.2365.

### Synthesis of N-(methyl-PEG4)-4-(3-isocyanopropyl-2-Phenol-1-oxy)-1,8-naphthalimide (ICPrc-2-Ph-O-NA; Structure 15)

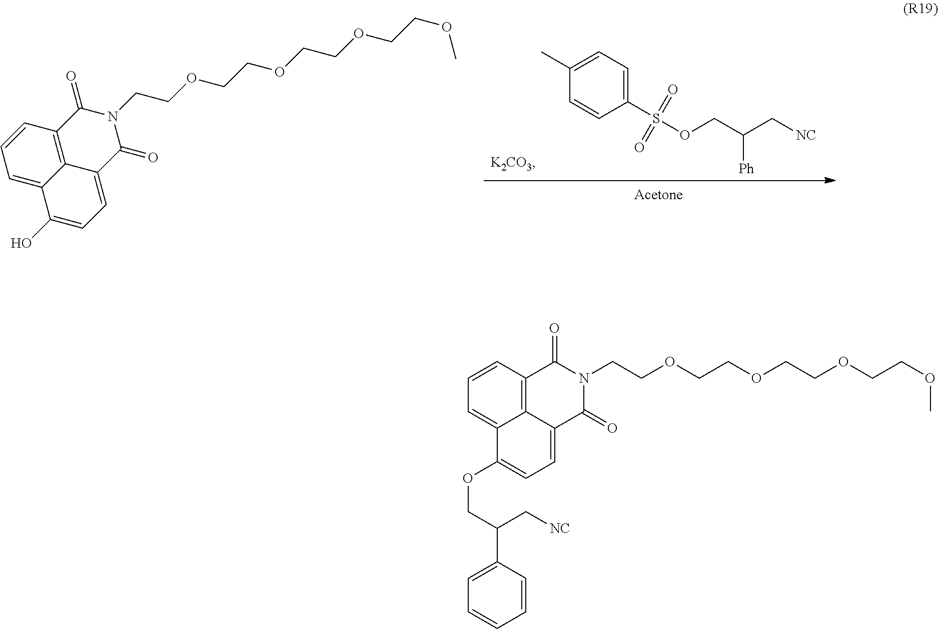

Reaction R19 was performed by adding anhydrous K₂CO₃ (22 mg, 0.16 mmol) to a mixture of N-(methyl-PEG4)-4-hydroxy-1,8-naphthalimide (41 mg, 0.1 mmol) in acetone (0.5 mL) and the mixture was allowed to stir at room temperature for 15 minutes. 3-isocyano-2-phenyl-1-tosylpropane (Structure 08; 60 mg, 0.2 mmol) dissolved in acetone (0.5 mL) was added dropwise and the reaction mixture was allowed to stir overnight at 50° C. for 12 h. The solution was cooled to room temperature, solid residues was removed by filtration, and organic layer was concentrated under reduced pressure and purified by column chromatography (DCM:MeOH=30:1, v/v) to afford N-(methyl-PEG4)-4-(3-isocyanopropyl-2-Phenol-1-oxy)-1,8-naphthalimide (ICPrc-2-Ph-O-NA; Structure 15) as an orange oil in a yield of 12 mg. (24%, R_{f}=0.3 in DCM:MeOH=20:1, v/v).

¹H NMR (400 MHz, CDCl₃) δ 8.61 (d, J=7.2 Hz, 1H), 8.52 (d, J=8.0 Hz, 1H), 8.47 (d, J=8.4 Hz, 1H), 7.72 (t, J=7.6 Hz, 1H), 7.45-7.37 (m, 5H), 7.06 (d, J=8.0 Hz, 1H), 4.58 (d, J=6.4 Hz, 2H), 4.43-4.40 (m, 2H), 4.04-4.00 (m, 2H), 3.80 (d, J=6.4 Hz, 3H), 3.70-3.67 (m, 4H), 3.62-3.56 (m, 8H), 3.52-3.49 (m, 2H), 3.35 (s, 3H), 3.15-3.09 (m, 1H). ¹³C NMR (100 MHz, CDCl₃) δ 164.3, 163.7, 159.1, 145.2, 136.7, 135.6, 133.2, 131.7, 130.0, 129.4, 129.2, 129.0, 128.4, 128.4, 128.2, 127.9, 127.6, 127.6, 126.2, 123.3, 122.5, 115.7, 106.0, 71.8, 70.5, 70.4, 70.0, 69.2, 69.0, 67.9, 44.4, 43.9, 21.6. HRMS (ESI): calcd. for C₃₁H₃₄N₂O₇ [M+Na]⁺ 569.2258, found 569.2295.

### Synthesis of N-(methyl-PEG4)-4-(3-isocyanopropyl-1-phenylcarbamoyl)amino-1,8-naphthalimide (ICPrc-3-Ph-NH-NA; Structure OX 16)

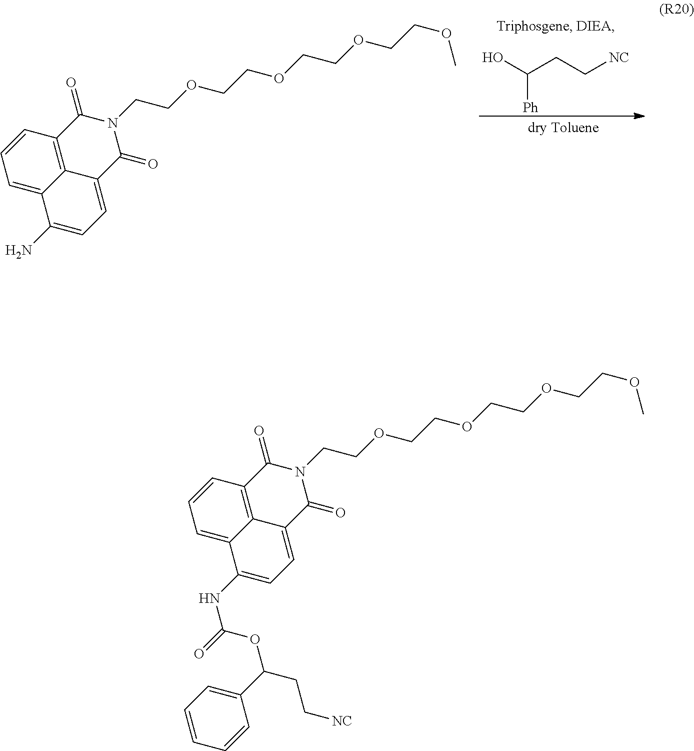

Reaction R20 was performed by adding dropwise a solution of triphosgene (39 mg, 0.13 mmol) in dry toluene (1 mL) to a mixture of N-(methyl-PEG4)-4-amino-1,8-naphthalimide (48 mg, 0.12 mmol) and DIEA (44 mg, 0.36 mmol) in dry toluene (2.5 mL). The solution was heated to reflux for 3 h. After cooling to room temperature, a solution of 3-isocyanide-1-phenylpropan-1-ol (39 mg, 0.24 mmol) in dry CH₂Cl₂ (3 mL) was added to the mixture and the reaction was stirred at room temperature for 12 h. Then the mixture was concentrated under reduced pressure and purified by column chromatography (DCM:MeOH=20:1, v/v) to afford N-(methyl-PEG4)-4-(3-isocyanopropyl-1-phenylcarbamoyl)amino-1,8-naphthalimide (ICPrc-3-Ph-NH-NA; Structure OX 16) as a yellow oil in a yield of 15 mg (35%, R_{f}=0.35 in EA:hexane=1:3, v/v).

¹H NMR (400 MHz, CDCl₃) δ 8.52 (t, J=8.0 Hz, 2H), 8.32-8.29 (m, 2H), 8.09 (brs, 1H), 7.73 (t, J=8.0 Hz, 1H), 7.59-7.49 (m, 5H), 6.13-6.09 (m, 1H), 4.52-4.49 (m, 2H), 3.97 (t, J=6.0 Hz, 2H), 3.87-3.85 (m, 2H), 3.78-3.68 (m, 6H), 3.63-3.59 (m, 5H), 3.55-3.53 (m, 2H), 3.42 (s, 3H), 2.63-2.37 (m, 2H). ¹³C NMR (100 MHz, CDCl₃) δ 163.9, 163.4, 157.0, 152.2, 138.8, 138.3, 132.1, 131.0, 128.8, 128.8, 128.3, 126.3, 126.2, 122.7, 117.3, 116.8, 74.7, 71.6, 70.5, 70.3, 70.3, 70.2, 70.0, 67.9, 58.8, 38.9, 38.2, 35.5, 29.5. HRMS (ESI): calcd. for C₃₂H₃₅N₃O₈ [M+Na]⁺ 612.2316, found 612.2371.

### Synthesis of 3-isocyanopropyl resorufin ether (ICPr-rsf, Structure 17)

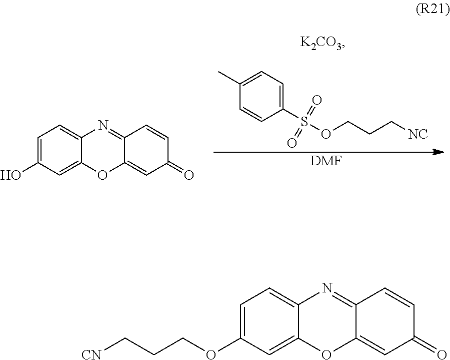

Reaction R21 was performed by adding anhydrous K₂CO₃ (156 mg, 1.13 mmol) to a solution of resorufin (80 mg, 0.38 mmol) in anhydrous DMF (10 mL). The mixture was heated to 80° C. under N₂ atmosphere and stirred for 15 minutes. 3-isocyano-1-tosylpropane (270 mg, 1.13 mmol) dissolved in anhydrous DMF (1 mL) was added dropwise and the reaction mixture was allowed to stir overnight at 80° C. (16 h). The solution was cooled to room temperature, solid residues were removed by filtration, and the filtrate was concentrated under reduced pressure. The residue was dissolved in DCM (3×100 mL), the organic layer was washed with water (2×100 mL) and brine (1×200 mL), dried with Mg₂SO₄, filtrated, and evaporated. The residue was purified by silica gel chromatography (DCM:MeOH=20:1, v/v, R_{f}=0.4) to afford Synthesis of 3-isocyanopropyl resorufin ether (ICPr-rsf, Structure 17) as a bright orange solid in a yield 90 mg (86%).

¹H NMR (400 MHz, DMSO-d₆) δ 7.77 (d, J=8.8 Hz, 1H), 7.52 (d, J=9.8 Hz, 1H), 7.14 (d, J=2.4 Hz, 1H), 7.06 (dd, J₁=8.8, J₂=2.4 Hz, 1H), 6.77 (dd, J₁=9.8 Hz, J₂=1.8 Hz, 1H), 6.25 (d, J=2.0 Hz, 1H), 4.21 (d, J=6.0 Hz, 2H), 3.68 (t, J=6.4 Hz, 2H), 2.09 (s, 2H). ¹³C NMR (100 MHz, DMSO-d₆) δ 185.8, 162.7, 156.8, 150.2, 145.7, 135.4, 134.2, 131.8, 128.5, 114.5, 106.1, 101.3, 65.8, 40.6, 40.4, 40.2, 40.0, 40.8, 39.5, 39.3, 38.7, 38.7, 38.6, 28.4.

### Synthesis of 7-(3-isocyanopropyl-1-oxy)-coumarin (ICPr-coum, Structure 18)

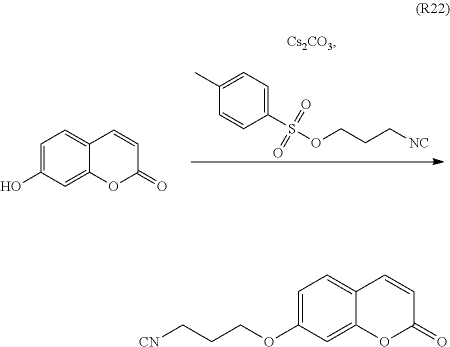

Reaction R22 was performed by adding anhydrous Cs₂CO₃ (147 mg, 0.45 mmol) to a solution of 7-hydroxycoumarin (umbelliferone, 50 mg, 0.30 mmol) in anhydrous DMF (1 mL) and the mixture was allowed to stir at room temperature for 15 minutes. 3-isocyano-1-tosylpropane (215 mg, 0.9 mmol) dissolved in anhydrous DMF (0.5 mL) was added dropwise and the reaction mixture was allowed to stir for 4 h at 60° C. The solution was cooled to room temperature, solid residues were removed by filtration, and the filtrate was concentrated under reduced pressure. The residue was dissolved in DCM (3×50 mL), the organic layer washed with water (2×50 mL) and brine (1×100 mL), dried with Mg₂SO₄, filtrated and evaporated. The residue was purified by silica gel chromatography (DCM:MeOH=20:1, v/v, R_{f}=0.25) to afford 7-(3-isocyanopropyl-1-oxy)-coumarin (ICPr-coum, Structure 18) as an off-white solid in a yield of 60 mg (87%).

¹H NMR (400 MHz, CDCl₃) δ 7.63 (d, J=9.6 Hz, 1H), 7.38 (d, J=8.6 Hz, 1H), 6.87-6.76 (m, 2H), 6.25 (d, J=9.6 Hz, 1H), 4.16 (t, J=5.6 Hz, 2H), 3.65 (t, J=6.2 Hz, 2H), 2.18 (s, 2H). ¹³C NMR (100 MHz, CDCl₃) δ 161.5, 161.0, 157.1, 155.8, 143.3, 128.9, 113.4, 112.9, 112.5, 101.6, 77.4, 77.1, 76.7, 64.1, 38.4, 38.3, 38.3, 29.3.

### Synthesis of N-(3-isocyanopropyl-1-carbamoyl)Mitomycin C (ICPrc-mmc; Structure 19)

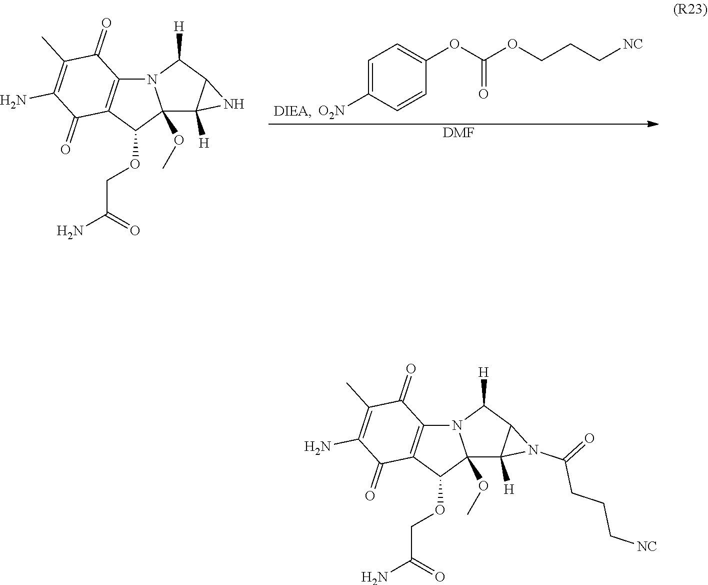

Reaction R23 was performed by adding DIEA (129 mg, 1 mmol) to a solution of 3-isocyanopropyl-1(4-nitrophenyl)carbonate (25 mg, 0.1 mmol) in dry DMF (0.5 mL) and the mixture was stirred for 30 min. Mitomycin C (41 mg, 0.12 mmol) was added and stirring was continued at 25° C. for 12 h. The mixture was diluted with CH₂C₂ (100 mL) and washed with H₂O (50 mL) and brine (2×50 mL). The organic phase was dried over Na₂SO₄, filtrated, and concentrated for purification by preparative-TLC (DCM:MeOH=15:1, v/v; R_{f}=0.25) to afford N-(3-isocyanopropyl-1-carbamoyl)Mitomycin C (ICPrc-mmc; Structure 19) as purple solid in a yield of 19 mg (45%).

¹H NMR (400 MHz, MeOD-d₄) δ 4.85-4.80 (m, 1H), 4.38 (d, J=13.2 Hz, 1H), 4.21 (t, J=6.4 Hz, 2H), 4.15 (t, J=10.8 Hz, 1H), 3.67-3.65 (m, 1H), 3.63 (d, J=4.8 Hz, 1H), 3.58-3.54 (m, 2H), 3.50-3.44 (m, 2H), 3.22 (s, 3H), 2.03-2.00 (m, 2H), 1.74 (s, 3H). ¹³C NMR (100 MHz, MeOD-d₄) δ 172.3, 170.8, 155.9, 152.3, 150.2, 144.2, 109.9, 104.5, 100.5, 98.5, 58.2, 56.3, 46.9, 46.7, 46.5, 46.3, 46.1, 43.6, 43.4, 38.2, 36.9, 34.7, 32.9, 22.9, 1.6.

### Synthesis of N-(3-isocyanopropyl-1-carbamoyl)doxorubicin (ICPrc-dox; Structure 20)

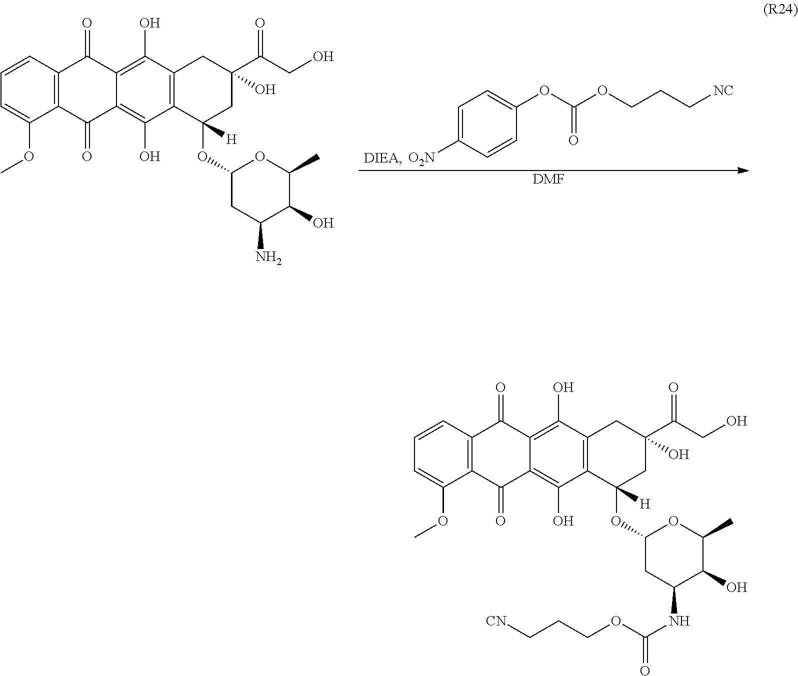

Reaction R24 was performed by adding doxorubicin hydrochloride (70 mg, 0.12 mmol) to a solution of 3-isocyanopropyl-1-(4-nitrophenyl)carbonate (25 mg, 0.1 mmol) and DIEA (129 mg, 1 mmol) in dry DMF (0.5 mL) and the mixture was stirred at 25° C. for 12 h. The mixture was diluted with CH₂Cl₂ (100 mL) and washed with H₂O (50 mL) and brine (2×50 mL). The organic phase was dried with Na₂SO₄, filtered, and concentrated for purification by preparative-TLC (DCM:MeOH=15:1, v/v; R_{f}=0.35) to afford N-(3-isocyanopropyl-1-carbamoyl)doxorubicin (ICPrc-dox; Structure 20) as red solid in a yield of 12 mg (22%).

¹H NMR (400 MHz, CDCl₃:MeOD-d₄=9:1, v/v) δ 7.97 (d, J=8.0 Hz, 1H), 7.73 (t, J=8.0 Hz, 1H), 7.35 (d, J=8.4 Hz, 1H), 5.58 (d, J=8.0 Hz, 1H), 5.42 (s, 1H), 5.21 (brs, 1H), 4.10-4.05 (m, 2H), 4.01 (s, 2H), 3.76 (brs, 1H), 3.54 (brs, 1H), 3.42 (brs, 1H), 3.22 (s, 6H), 2.97 (d, J=18.8 Hz, 1H), 2.29 (d, J=14.4 Hz, 1H), 2.11-2.07 (m, 1H), 1.89 (brs, 2H), 1.74 (brs, 2H), 1.21 (d, J=6.4 Hz, 2H), 1.18 (s, 1H).

¹³C NMR (100 MHz, CDCl₃:MeOD-d₄=9:1, v/v) δ 213.8, 187.1, 186.7, 161.0, 155.8, 155.6, 155.2, 135.8, 135.4, 133.7, 133.5, 120.7, 119.8, 118.5, 111.5, 111.3, 100.7, 76.3, 69.4, 68.9, 67.4, 65.2, 61.0, 56.5, 49.4, 49.2, 49.1, 48.9, 48.7, 46.9, 38.6, 38.6, 38.5, 35.7, 33.6, 29.9, 28.7, 16.6.

### Synthesis of N-(3-isocyanopropyl-1-carbamoyl)mexiletine (ICPrc-mex, Structure 21)

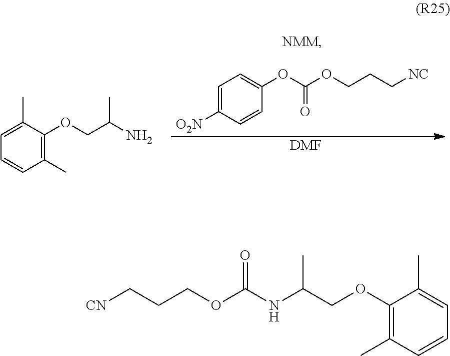

Reaction R25 was performed by adding mexiletine hydrochloride (32 mg, 0.15 mmol) to a solution of 3-isocyanopropyl-1-(4-nitrophenyl)carbonate (15 mg, 0.05 mmol) and N-methylmorpholine (51 mg, 0.5 mmol) in dry DMF (0.2 mL) and the mixture was stirred at 25° C. for 12 h. The mixture was diluted with CH₂Cl₂ (100 mL) and washed with H₂O (50 mL) and brine (2×50 mL). The organic phase was dried with Mg₂SO₄, filtered, and concentrated for purification by preparative-TLC (DCM:MeOH=20:1 v/v; R_{f}=0.5) to afford N-(3-isocyanopropyl-1-carbamoyl)mexiletine (ICPrc-mex, Structure 21) as an off-white oil in a yield of 1.5 mg (10%).

¹H NMR (400 MHz, MeOD-d₄) δ 7.02 (d, J=7.4 Hz, 2H), 6.96-6.91 (m, 1H), 3.91-3.77 (m, 3H), 3.74-3.64 (m, 2H), 2.29 (s, 6H), 1.66-1.53 (m, 2H), 1.42 (d, J=6.7 Hz, 3H).

### Synthesis of N-(3-isocyanopropyl-1-carbamoyl)SN-38 (ICPr-SN-38, Structure 22)

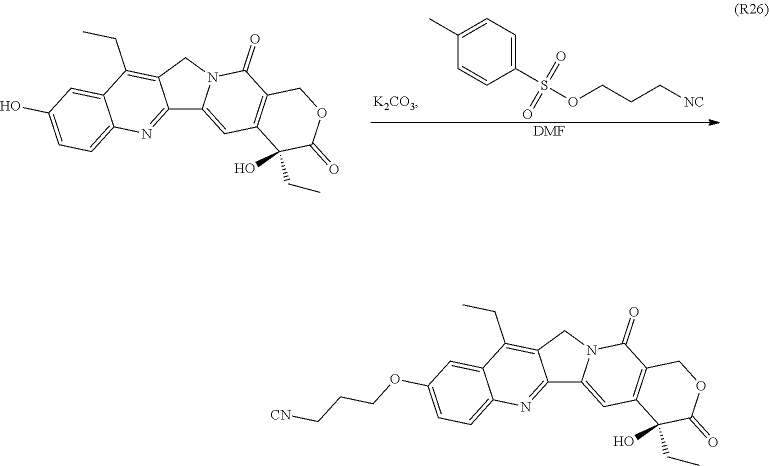

Reaction R26 was performed by adding K₂CO₃ (21 mg, 0.15 mmol) to a solution of SN-38 (20 mg, 0.05 mmol) in dry DMF (1 mL). The mixture was heated to 80° C. and stirred for 15 minutes under N₂ atmosphere. 3-isocyano-1-tosylpropane (47 mg, 0.20 mmol) dissolved in anhydrous DMF (1 mL) was added dropwise and the reaction mixture was allowed to stir overnight at 80° C. (16 h). The solution was cooled to room temperature, solid were residues removed by filtration, and the filtrate was concentrated under reduced pressure. The residue was dissolved in DCM (3×100 mL), washed with water (2×100 mL) and brine (200 mL) and purified by silica gel chromatography to afford N-(3-isocyanopropyl-1-carbamoyl)SN-38 (ICPr-SN-38, Structure 22).

¹H NMR (400 MHz, CDCl₃) δ 8.15 (d, J=9.2 Hz, 1H), 7.61 (s, 1H), 7.46 (dd, J=9.2, 2.6 Hz, 1H), 7.35 (d, J=2.6 Hz, 1H), 5.76 (d, J=16.2 Hz, 1H), 5.31 (d, J=16.2 Hz, 1H), 4.32 (t, J=5.6 Hz, 2H), 3.74 (t, J=6.2 Hz, 2H), 3.16 (dd, J=15.2, 7.6 Hz, 2H), 2.27 (s, 2H), 1.95-1.82 (m, 2H), 1.41 (t, J=7.8 Hz, 3H), 1.34 (s, 2H), 1.04 (t, J=7.4 Hz, 2H).

### Synthesis of 6-(3-isocyanopropyl)mercaptopurine (ICPr-mp; Structure 23)

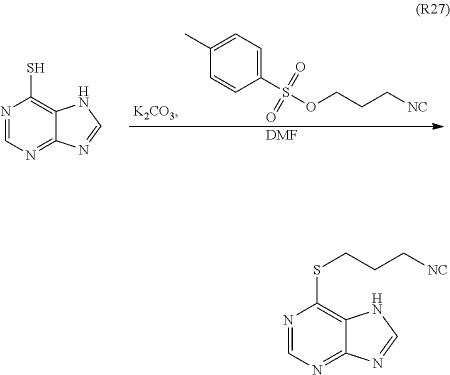

Reaction R27 was performed by adding K₂CO₃ (32 mg, 0.23 mmol) to a solution of 6-mercaptopurine (50 mg, 0.23 mmol) in dry DMF (1 mL) and the mixture was allowed to stir at room temperature for 30 minutes. 3-isocyano-1-tosylpropane (66 mg, 0.28 mmol) in dry DMF (0.1 mL) was added dropwise and the reaction mixture was allowed to stir at room temperature for 2 hours. The mixture was concentrated under reduced pressure, dissolved in DCM and solid residues were eliminated by filtration. The product was purified by preparative TLC (DCM:MeOH=10:1, v/v, R_{f}=0.40) to afford 6-(3-isocyanopropyl)mercaptopurine (ICPr-mp; Structure 23) as a white solid in a yield of 47 mg. (93%) H NMR (400 MHz, DMSO-d₆) δ 8.67 (s, 1H), 8.43 (s, 1H), 3.68-3.58 (m, 2H), 3.41 (t, J=7.2 Hz, 2H), 2.05 (s, 2H). ¹³C NMR (100 MHz, DMSO-d₆) δ 161.5, 157.9, 156.7, 151.9, 143.9, 129.7, 40.7, 29.2, 25.2.

### Synthesis of 2-(3-isocyanopropyl-2-phenyl)Naphthalenethiol (Structure 24)

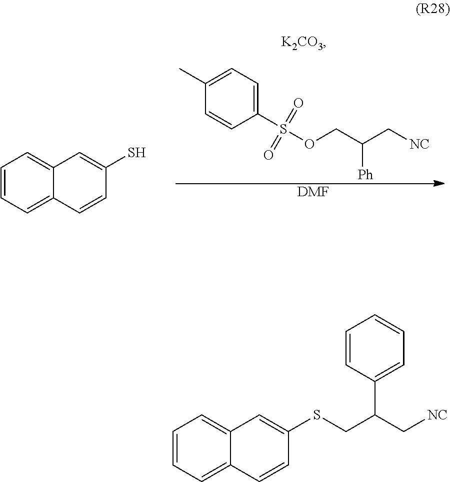

Reaction R28 was performed by To a mixture of 2-Naphthalenethiol (6 mg, 0.033 mmol) in DMF (0.1 mL) was added anhydrous K₂CO₃ (17 mg, 0.12 mmol) and the mixture was allowed to stir at room temperature for 15 minutes. Compound 5 (10 mg, 0.03 mmol) dissolved in DMF (0.1 mL) was added dropwise and the reaction mixture was allowed to stir overnight at r.t for 12 h. The solution was cooled to room temperature, solid residues was removed by filtration, and organic layer was concentrated under reduced pressure and purified by column chromatography (Hexane:EA=10:1, v/v) to afford 2-(3-isocyanopropyl-2-phenyl)Naphthalenethiol (Structure 24) as an orange oil in a yield of 8 mg. (20%, R_{f}=0.3 in Hexane:EA=10:1, v/v).

¹H NMR (400 MHz, CDCl₃) δ 7.82-7.76 (m, 4H), 7.50-7.31 (m, 6H), 7.22 (d, J=7.2 Hz, 2H), 3.87-3.76 (m, 2H), 3.43-3.40 (m, 2H), 3.17 (brs, 1H). ¹³C NMR (100 MHz, CDCl₃) δ 139.1, 133.6, 132.2, 132.0, 128.9, 128.8, 128.0, 127.7, 127.5, 127.5, 127.1, 126.7, 126.0, 45.8, 44.0, 36.2, 31.9, 29.7, 22.7. HRMS (ESI): calcd. for C₂₀H₁₇NS [M+K]⁺ 342.0713, found 342.0935 or need EI?.

### Example Procedures: Tetrazine Synthesis

### Synthesis of 6-(6-(pyridin-2-yl)-1,4-dihydro-1,2,4,5-tetrazin-3-yl)pyridin-3-amine (Structure 27)

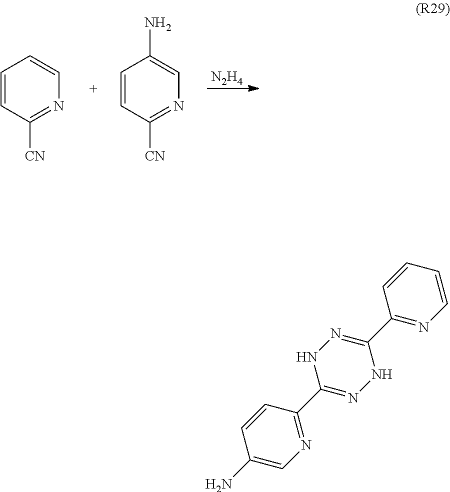

Reaction R29 was performed by adding hydrazine monohydrate (6 mL) to a mixture of 2-cyanopyridine (3.1 g, 30 mmol) and 5-amino-2-cyanopyridine (3.5 g, 30 mmol) over a period of 30 min and heated to reflux at 90° C. for 12 h. The mixture was diluted with CH₂Cl₂ (500 mL) and washed with H₂O (150 mL) and brine (2×150 mL). The organic phase was dried with Na₂SO₄, filtered, and concentrated for purification by column chromatography (Hexane:Acetone=1:1, v/v; R_{f}=0.35) to afford 6-(6-(pyridin-2-yl)-1,4-dihydro-1,2,4,5-tetrazin-3-yl)pyridin-3-amine (Structure 27) as yellow powder in a yield of 1.84 g (25%).

¹H NMR (400 MHz, DMSO-d₆) δ 8.70 (s, 1H), 8.65 (s, 1H), 8.63 (d, J=4.0 Hz, 1H), 7.97-7.91 (s, 3H), 7.65 (d, J=6.8 Hz, 1H), 7.52 (d, J=4.8 Hz, 1H), 7.00 (dd, J₁=2.0 Hz, J₂=6.8 Hz, 1H), 5.88 (s, 2H). The ¹H NMR data agreed with the reported spectrum of this compound.^{[3]}

### Synthesis of 6-(6-(pyridin-2-yl)-1,2,4,5-tetrazin-3-yl)pyridin-3-amine (Structure 28)

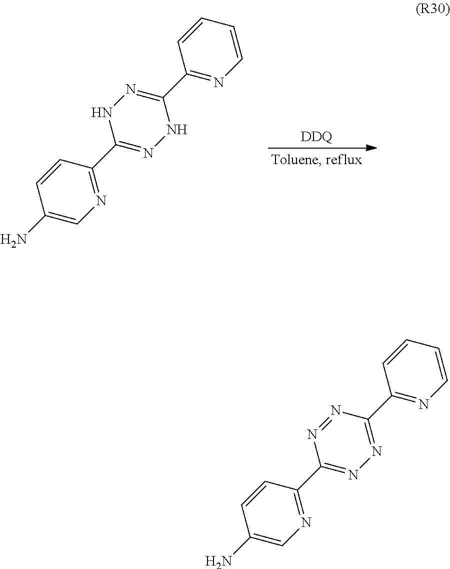

Reaction 30 was performed by adding 2,3-dichloro-5,6-dicyano-p-benzoquinone (3.5 g, 15 mmol) to a solution of 6-(6-(pyridin-2-yl)-1,4-dihydro-1,2,4,5-tetrazin-3-yl)pyridin-3-amine (1.78 g, 7 mmol) in dry toluene (50 mL) and reaction was heated to reflux for 12 h under a nitrogen atmosphere. The mixture was diluted with CH₂Cl₂ (200 mL) and washed with H₂O (150 mL) and brine (2×150 mL). The organic phase was dried with Na₂SO₄, filtered, and concentrated for purification by column chromatography (Hexane:Acetone=10:1, v/v; R_{f}=0.25) to afford 6-(6-(pyridin-2-yl)-1,2,4,5-tetrazin-3-yl)pyridin-3-amine (Structure 28) as pink powder in a yield of 875 mg (50%).

¹H NMR (400 MHz, DMSO-d₆) δ 8.88 (d, J=4.4 Hz, 1H), 8.51 (d, J=8.0 Hz, 1H), 8.36 (d, J=8.8 Hz, 1H), 8.21 (d, J=2.4 Hz, 1H), 8.13-8.09 (m, 1H), 7.67 (t, J=5.6 Hz, 1H), 7.11 (dd, J₁=2.8 Hz, J₂=8.8 Hz, 1H). The ¹H NMR data agreed with the reported spectrum of this compound.

### Synthesis of N-(methyl-PEG4)-6-(6-(pyridin-2-yl)-1,2,4,5-tetrazin-3-yl)pyridin (PEG-DPTz; Structure 29)

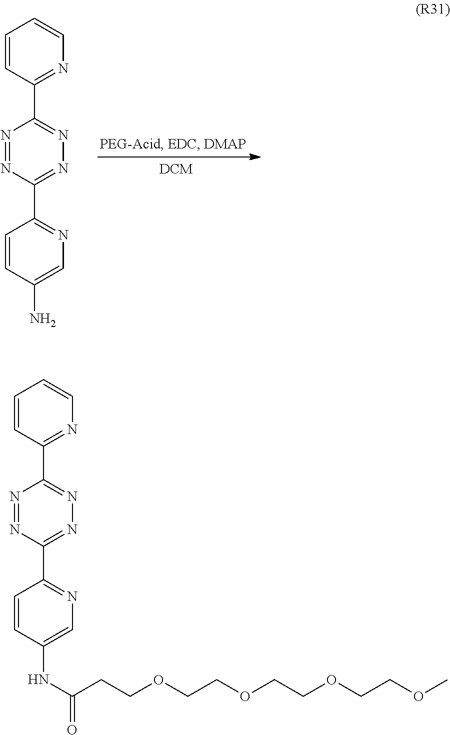 [SMILES: Nc1ccc(-c2nnc(-c3ccccn3)nn2)nc1 ; COCCOCCOCCOCCC(=O)Nc1ccc(-c2nnc(-c3ccccn3)nn2)nc1]

Reaction 31 was performed by adding a mixture of 6-(6-(pyridin-2-yl)-1,2,4,5-tetrazin-3-yl)pyridin-3-amine (83 mg, 0.33 mmol) and DMAP (51 mg, 0.42 mmol) in CH₂Cl₂ (2 mL) to a solution of m-PEG4 acid (71 mg, 0.3 mmol) in CH₂Cl₂ (2 mL) at 0° C. After the solution was kept for another 30 min at 0° C., EDC HCl (103 mg, 1.2 mmol) was slowly added. The ice-water bath was removed, and the mixture was stirred for 6 h at room temperature. Then the mixture was purified by preparative TLC (EA:MeOH=20:1, v/v; R_{f}=0.25) to afford N-(methyl-PEG4)-6-(6-(pyridin-2-yl)-1,2,4,5-tetrazin-3-yl)pyridin (PEG-DPTz; Structure 29) as a dark pink oil in a yield of 28 mg (20%).

¹H NMR (400 MHz, MeOD-d₄) δ 9.06 (d, J=2.4 Hz, 1H), 8.88 (d, J=4.8 Hz, 1H), 8.78 (t, J=8.4 Hz, 2H), 8.50 (dd, J₁=2.4 Hz, J₂=8.4 Hz, 1H), 8.21-8.17 (m, 1H), 7.76-7.74 (m, 1H), 3.86 (t, J=6.0 Hz, 2H), 3.66-3.62 (m, 6H), 3.60-3.57 (m, 4H), 3.50-3.48 (m, 2H), 3.31 (s, 3H), 2.75-2.72 (m, 2H).

¹³C NMR (100 MHz, MeOD-d₄) δ 171.7, 163.2, 163.1, 149.9, 149.7, 143.9, 141.3, 138.8, 138.3, 127.1, 126.8, 124.8, 124.2, 71.5, 70.1, 70.0, 69.9, 66.5, 37.2.

### Synthesis of 4-(6-methyl-1,2,4,5-tetrazin-3-yl)benzoic acid (Structure 30)

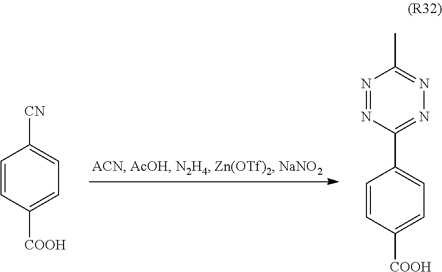 [SMILES: N#Cc1ccc(C(=O)O)cc1 ; Cc1nnc(-c2ccc(C(=O)O)cc2)nn1]

Reaction 32 was performed by mixing 4-Cyanobenzoic acid (1.47 g, 10 mmol), zinc trifluoromethanesulfonate (1.84 g, 5.1 mmol) and acetonitrile (6 mL) together and stirring for 30 min. Hydrazine monohydrate (24.3 mL) was added to the mixture under a nitrogen atmosphere over a period of 1 h. The mixture was warmed up to 60° C. and stirred for 24 h. The mixture was cooled to room temperature and a saturated sodium nitrite solution (50 mL) was added and the mixture was stirred for 30 min. 1 M HCl was added over a period of 30 min to the mixture to adjust solution pH 2. (Caution: Nitrogen oxide gas was released). The mixture was diluted with CH₂Cl₂ (500 mL) and washed with H₂O (200 mL) and brine (2×150 mL). The organic phase was dried with Na₂SO₄, filtered, and concentrated by evaporation for purification by column chromatography (100% Acetone, v/v; R_{f}=0.55) to afford 4-(6-methyl-1,2,4,5-tetrazin-3-yl)benzoic acid (Structure 30) as an pink powder in a yield of 1.32 g (61%).

¹H NMR (400 MHz, DMSO-d₆) δ 8.57 (d, J=8.8 Hz, 2H), 8.19 (d, J=8.8 Hz, 2H), 3.01 (s, 3H). The ¹H NMR data agreed with the reported spectrum of this compound.^{[4]}

### Synthesis of 4-(6-methyl-1,2,4,5-tetrazin-3-yl)benzoic N-Hydroxysuccinimide (Structure 31)

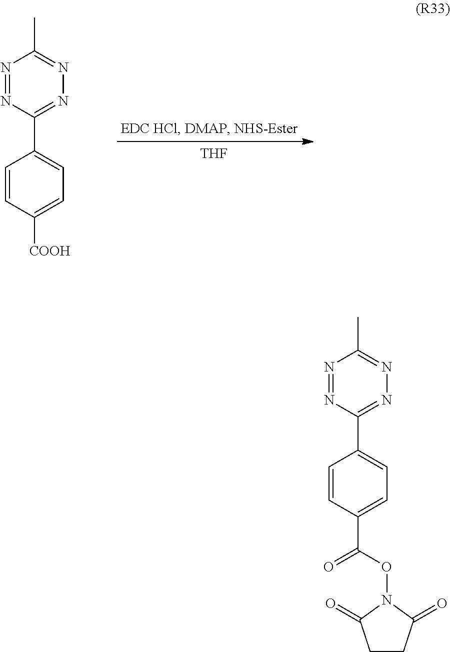 [SMILES: Cc1nnc(-c2ccc(C(=O)O)cc2)nn1 ; Cc1nnc(-c2ccc(C(=O)ON3C(=O)CCC3=O)cc2)nn1 ; "THF"]

Reaction 33 was performed by adding EDC HCl (23 mg, 0.12 mmol), NHS (14 mg, 0.12 mmol) and DMAP (5 mg, cat) to a solution of 4-(6-methyl-1,2,4,5-tetrazin-3-yl)benzoic acid (Structure 30) (21.6 mg, 0.1 mmol) in THE (1 mL) at 0° C. on ice and the reaction mixture was stirred at room temperature for 12 h. The mixture was diluted with CH₂Cl₂ (20 mL) and washed with H₂O (10 mL) and brine (2×10 mL). The organic phase was dried with Na₂SO₄, filtered, and concentrated for purification by column chromatography (DCM:MeOH=20:1, v/v; R_{f}=0.15) to afford the desired compound 4-(6-methyl-1,2,4,5-tetrazin-3-yl)benzoic N-Hydroxysuccinimide (Structure 31) as pink powder in a yield of 16 mg (52%).

¹H NMR (400 MHz, DMSO-d₆) δ 8.75 (d, J=8.4 Hz, 2H), 8.34 (d, J=8.4 Hz, 2H), 3.14 (s, 3H), 2.94 (brs, 5H). The ¹H NMR data agreed with the reported spectrum of this compound.

### Synthesis of 3,5-di(pyridine-2-yl)-1H-pyrazol-4-amine (DPPA; Structure 32)

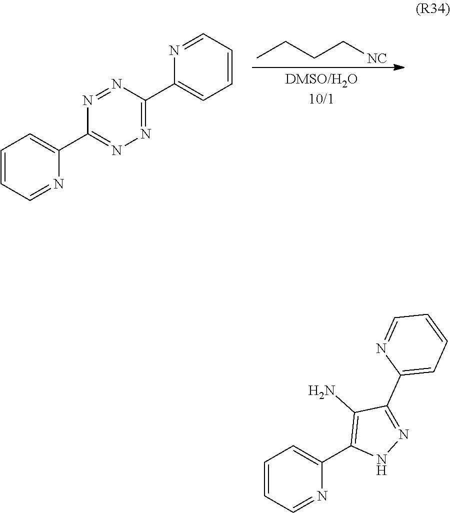

Reaction 34 was performed by adding n-butyl isocyanide (77 mg, 0.93 mmol) to a solution of 3,6-di(pyridin-2-yl)-1,2,4,5-tetrazine (110 mg, 0.47 mmol) in DMSO:H₂O (10:1, 2 mL) with stirring at room temperature for 2 h. The solution was left to stand at ambient temperature and subjected to a low stream of nitrogen gas until a precipitate formed. The precipitate was collected and dried to give 3,5-di(pyridine-2-yl)-1H-pyrazol-4-amine (DPPA; Structure 32) (90 mg, 81% yield) as golden crystals.

¹H NMR (400 MHz, DMSO-d₆) δ 13.16 (s, 1H), 8.60 (dd, J₁=4.6 Hz, J₂=9.2 Hz, 2H), 7.98 (d, J=8.0 Hz, 1H), 7.89-7.76 (m, 3H), 7.27-7.17 (m, 2H), 6.01 (s, 2H). The ¹H NMR data agreed with the reported spectrum of this compound.

Synthesis of Tetrazine modified Tentagel

25 mg Tenta Gel (TentaGel® HL—NH₂, 75 μm, Rapp-Polymere) was swollen in DCM for 30 min and rinsed with DMF for 15 min. A mixture of 4-(6-methyl-1,2,4,5-tetrazin-3-yl)benzoic N-Hydroxysuccinimide (Structure 31) (8 mg, 0.025 mmol), DIEA (31 mg, 0.25 mmol) in DMF (2 mL) was added into the resin. The reaction is maintained at room temperature for 12 h. The resin was washed with DMF (2×5 mL) and DCM (2×5 mL) and dried through vacuum line.

### Extent of Labeling Assay

The extent of labeling was approximately 75%, as determined by sequential reaction with N-succinimidyl 3-(2-pyridyldithio) propionate (SPDP; TCI) and dithiothreitol to liberate pyridine-2-thione.

### Materials and Instrumentation

All chemical reagents and solvents were obtained from commercial sources (Sigma-Aldrich, Alfa-Aesar, Combi-Blocks, Acros-Organic, TCI) and used without further purification. Thin-layer chromatography (TLC) analysis was carried out to monitor the process of reactions. Purification of compounds was performed by column chromatography with silica gel 300-400 mesh. ¹H NMR and ¹³C NMR spectra were recorded on a Varian Mercury-400 or Varian Inova-500 spectrometer as indicated with chemical shifts expressed as ppm (in CDCl₃, MeOD-d₄ or DMSO-d₆) using Me₄Si (TMS) as internal standard.

High-resolution Mass Spectra were measured by the University of California, Riverside Chemistry Mass Spectrometry Facility. All analytical preparative HPLCs were performed on a Dionex Ultimate3000 equipped with autosampler, diode array detector and robotic fraction collector (Dionex Thermo Scientific, USA) using a LUNA C18 column (5 μM, 250×10 mm, Phenomenex, USA) or LUNA C18 column (5 μM, 150×2.0 mm, Phenomenex, USA).

UV-VIS photospectrometic kinetic measurements were performed on a microplate reader SpectramaX M5 (Molecular Device, USA) in 96-well plates or 1 mL quartz cuvette. Cell proliferation assays were performed on an Envision 2104 Multilabel Reader (PerkinElmer, USA). Zebrafish Imaging and Efficiency Test (Heart Rate Reduction) were performed with Zeiss SteREO Discovery.V8 microscope (Zeiss, Jena, Germany) fitted with a PentaFluor S 120 vertical illuminator and coupled with an Xcite Series 120PC light source.

What is claimed is:

1. A bioorthogonal molecule, comprising:
a molecule having a structure according to: 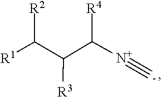
where R², R³, and R⁴ are independently selected from H, a substituted or unsubstituted C₁-C₄ alkyl or alkylene group, a substituted or unsubstituted aryl, COOR⁹, COR⁹, CONR⁹R¹⁰, CN, CF₃, SO₂R⁹, or a tether molecule;
R¹ is —R⁵, —OCOR⁶, —COR⁷, or —R⁸;
R⁵ is —R⁸, —OH, or tosyl;
R⁶ is a nitrophenyl ether or —R⁸;
R⁷ is —R⁸;
R⁸ is a payload or a molecular linker to a payload; and
R⁹ and R¹⁰ are independently selected from H or a substituted or unsubstituted C₁-C₄ alkyl or alkene group.
